SEQUENCE LISTING (40 sequences)

SEQ ID NO: 1 - DNA, 19 bp, synthetic construct
  source 1..19: mol_type = other DNA
  misc_feature 1..19: note = primer
gcacgtccacgatcagtca

SEQ ID NO: 2 - DNA, 20 bp, synthetic construct
  source 1..20: mol_type = other DNA
  misc_feature 1..20: note = primer
cctttccccttcccccattc

SEQ ID NO: 3 - DNA, 47 bp, synthetic construct
  source 1..47: mol_type = other DNA
  misc_feature 1..47: note = primer
aaatctagaggtggtggtggttccggtgacgggtccggggaggagct

SEQ ID NO: 4 - DNA, 31 bp, synthetic construct
  source 1..31: mol_type = other DNA
  misc_feature 1..31: note = primer
tttgcggccgctcagcccatcttcttccaga

SEQ ID NO: 5 - DNA, 25 bp, synthetic construct
  source 1..25: mol_type = other DNA
  misc_feature 1..25: note = primer
gaattctttacgagggtaggaagtg

SEQ ID NO: 6 - DNA, 24 bp, synthetic construct
  source 1..24: mol_type = other DNA
  misc_feature 1..24: note = primer
ggatccagatctctgcagcatatg

SEQ ID NO: 7 - DNA, 39 bp, synthetic construct
  source 1..39: mol_type = other DNA
  misc_feature 1..39: note = primer
gcagagatctggatcctcagcccatcttcttccagatgg

SEQ ID NO: 8 - DNA, 37 bp, synthetic construct
  source 1..37: mol_type = other DNA
  misc_feature 1..37: note = primer
ccctcgtaaagaattcatggacgggtccggggagcag

SEQ ID NO: 9 - DNA, 43 bp, synthetic construct
  source 1..43: mol_type = other DNA
  misc_feature 1..43: note = primer
gtaaatctccagaggccgttttgtggaactactataactcgag

SEQ ID NO: 10 - DNA, 40 bp, synthetic construct
  source 1..40: mol_type = other DNA
  misc_feature 1..40: note = primer
atcccctgattctgtcatgcctgctattgtcttcccaatc

SEQ ID NO: 11 - DNA, 23 bp, synthetic construct
  source 1..23: mol_type = other DNA
  misc_feature 1..23: note = primer
acagaatcaggggataacgcagg

SEQ ID NO: 12 - DNA, 23 bp, synthetic construct
  source 1..23: mol_type = other DNA
  misc_feature 1..23: note = primer
cctctggagatttacccggggag

SEQ ID NO: 13 - DNA, 38 bp, synthetic construct
  source 1..38: mol_type = other DNA
  misc_feature 1..38: note = primer
atagatatcgtcgactccgcgcacatttccccgaaaag

SEQ ID NO: 14 - DNA, 38 bp, synthetic construct
  source 1..38: mol_type = other DNA
  misc_feature 1..38: note = primer
agtaacaaaggatcccgagggagcttccagggggaaac

SEQ ID NO: 15 - DNA, 24 bp, synthetic construct
  source 1..24: mol_type = other DNA
  misc_feature 1..24: note = primer
ggatcctttgttactttatagaag

SEQ ID NO: 16 - DNA, 23 bp, synthetic construct
  source 1..23: mol_type = other DNA
  misc_feature 1..23: note = primer
atgtcgacgatatctataacaag

SEQ ID NO: 17 - DNA, 23 bp, synthetic construct
  source 1..23: mol_type = other DNA
ggctaaagagttgaacgcaaagg

SEQ ID NO: 18 - DNA, 27 bp, synthetic construct
  source 1..27: mol_type = other DNA
  misc_feature 1..27: note = primer
ccaaatgaacacagtcaaggctgaatc

SEQ ID NO: 19 - DNA, 27 bp, synthetic construct
  source 1..27: mol_type = other DNA
  misc_feature 1..27: note = primer
agaatcaatgcttccagtgctaccaaa

SEQ ID NO: 20 - DNA, 27 bp, synthetic construct
  source 1..27: mol_type = other DNA
  misc_feature 1..27: note = primer
ggctttcaccgtagttcactggagact

SEQ ID NO: 21 - DNA, 27 bp, synthetic construct
  source 1..27: mol_type = other DNA
  misc_feature 1..27: note = primer
cacagtgactcttgtttccctctccca

SEQ ID NO: 22 - DNA, 22 bp, synthetic construct
  source 1..22: mol_type = other DNA
  misc_feature 1..22: note = primer
agggagctgcagtggagtaggc

SEQ ID NO: 23 - DNA, 22 bp, synthetic construct
  source 1..22: mol_type = other DNA
  misc_feature 1..22: note = primer
gacaacgcccacacaccaggtt

SEQ ID NO: 24 - DNA, 26 bp, synthetic construct
  source 1..26: mol_type = other DNA
  misc_feature 1..26: note = primer
atgggtgtatgggaagaggggagaga

SEQ ID NO: 25 - DNA, 30 bp, synthetic construct
  source 1..30: mol_type = other DNA
  misc_feature 1..30: note = primer
ttcctaccctcgtaaagaattcatggacgg

SEQ ID NO: 26 - DNA, 25 bp, synthetic construct
  source 1..25: mol_type = other DNA
  misc_feature 1..25: note = primer
gtgccactcccactgtcctttccta

SEQ ID NO: 27 - DNA, 31 bp, synthetic construct
  source 1..31: mol_type = other DNA
  misc_feature 1..31: note = primer
aatcatgttaaactggaggagggtagtggtg

SEQ ID NO: 28 - DNA, 23 bp, synthetic construct
  source 1..23: mol_type = other DNA
tggcagctcactcccaactctgg

SEQ ID NO: 29 - DNA, 42 bp, synthetic construct
  source 1..42: mol_type = other DNA
tgtgtgggcgttgtcctctggagtctctaactccagttccat

SEQ ID NO: 30 - DNA, 42 bp, synthetic construct
  source 1..42: mol_type = other DNA
ccactgcagctccctttgggagtgagctgccatgtacattct

SEQ ID NO: 31 - DNA, 22 bp, synthetic construct
  source 1..22: mol_type = other DNA
agggagctgcagtggagtaggc

SEQ ID NO: 32 - DNA, 22 bp, synthetic construct
  source 1..22: mol_type = other DNA
gacaacgcccacacaccaggtt

SEQ ID NO: 33 - DNA, 27 bp, synthetic construct
  source 1..27: mol_type = other DNA
ggcctaaagtttatcacctgctagctg

SEQ ID NO: 34 - DNA, 27 bp, synthetic construct
  source 1..27: mol_type = other DNA
cagatgctagaaagactggagttgcag

SEQ ID NO: 35 - DNA, 27 bp, synthetic construct
  source 1..27: mol_type = other DNA
ggatccactagttctagagcggccgat

SEQ ID NO: 36 - DNA, 27 bp, synthetic construct
  source 1..27: mol_type = other DNA
gaaccaaccagacgaccagaccctaga

SEQ ID NO: 37 - DNA, 27 bp, synthetic construct
  source 1..27: mol_type = other DNA
ggcctaaagtttatcacctgctagctg

SEQ ID NO: 38 - DNA, 30 bp, synthetic construct; identical to SEQ ID NO: 25
  source 1..30: mol_type = other DNA

SEQ ID NO: 39 - DNA, 27 bp, synthetic construct
  source 1..27: mol_type = other DNA
cttgacatgctccccgggtaaatctcc

SEQ ID NO: 40 - DNA, 27 bp, synthetic construct
  source 1..27: mol_type = other DNA
gaaccaaccagacgaccagaccctaga

(19) United States
(12) Patent Application Publication
(10) Pub. No.: US 2025/0120375 A1
(43) Pub. Date: Apr. 17, 2025

(54) TRANSGENIC NON-HUMAN ANIMAL GIVING BIRTH TO INDIVIDUALS OF ONLY ONE SEX, AND METHODS FOR PRODUCING SAME

(71) Applicant: Kyoto University, Kyoto (JP)

(72) Inventors: Naojiro MINAMI, Kyoto (JP); Takuto YAMAMOTO, Kyoto (JP); Masahiro SAKATA, Kyoto (JP); Yuzuki GOTO, Kyoto (JP)

(73) Assignee: Kyoto University, Kyoto (JP)

(21) Appl. No.: 18/910,714

(22) Filed: Oct. 9, 2024

(30) Foreign Application Priority Data
Oct. 11, 2023 (JP) 2023-176171

(51) Int. Cl. A01K 67/0278; C12N 5/071

(52) U.S. Cl. CPC A01K 67/0278; C12N 5/0612; A01K 2217/203

(57) ABSTRACT

Technical Problem The purpose of the present invention is to provide a method which enables sex selection in a non-human animal, and enables a coming individual to have no recombination gene, and a transgenic non-human animal giving birth to individuals of only one sex, which is used in the method.

Solution to Problem The present disclosure relates to a male transgenic non-human animal in which one of an X chromosome and a Y chromosome comprises an exogenous embryonic death induction gene operatively connected to a drug-responsive promoter. The present disclosure also includes a method for producing a male transgenic non-human animal for sex selection.

### TECHNICAL FIELD

The present invention relates to a transgenic non-human animal giving birth to individuals of only one sex, and a method for producing the transgenic non-human animal.

### BACKGROUND ART

Control of the sex ratio of domestic animals which are industrial animals enables selection of a sex suiting a need, for example, the female in cows and the male in beef cattle, and can be expected to give a very large economic benefit to livestock operations. In the case of pigs, males that have reached a sexual maturity stage have strong odor, and thus are not suitable as edible meat, and therefore, male pigs are fattened after being castrated several weeks after the birth. However, the castration requires an enormous amount of labor, and has become a social problem involving, for example, criticism drawn from animal welfare organizations. In addition, in the case of experimental animals, the numbers of males and females may be different depending on a purpose of study, and if the sex ratio of animals can be controlled, necessity to make an adjustment at birth or a number adjustment after growth is eliminated, so that there is a major advantage that experimental animals can be effectively utilized. In the pet industry which has expanded its market in recent years, selection of the sex is very useful.

Regarding sex selection, sex determination on fertilized eggs using a genetic diagnosis technique (NPL 1: Alfieri et al., 2003) and sex determination by separation of sperm based on the amount of DNA (NPL 2: Johnson, 2000) are known. However, they are considered to hardly spread because time and expensive equipment are required for extraction of fertilized eggs, gene amplification by PCR and subsequent transplantation of fertilized eggs in the case of the former and for separation of sperm with a cell separation apparatus, freezing and the like after extraction of the sperm in the case of the latter. There is a FISH method in which chromosomes of cells are fluorescently stained (NPL 3: Lee et al., 2004), but the method also involves physically isolating and using a part of fertilized eggs, which causes deterioration of the viability, shelf life and conception rate of fertilized eggs. Further, the use of a cell surface histocompatibility Y (H-Y) antigen has reported to improve the accuracy of sex determination on bovine embryos (NPL 4: Veerhuis et al. 1994), but it has also been reported that this method is not reproducible (NPL 5: Bredbacka, 1998). In subsequent studies, an embryo biopsy (NPL 6: Harper et al., 1994) and a PCR-based assay of a sex chromosome-related gene have been used (NPL 7: HuaBin et al., 2012), but these methods have a disadvantage that a lot of labor is required in general, errors are likely to occur, and development of normal embryos may be impaired. Another method for selecting the sex utilizes the fact that when a GFP gene is knocked in onto a Y chromosome, fluorescence is observed only in male fertilized eggs (NPL 8: Zhao et al., 2019), but this method also has a problem that extraction of fertilized eggs and transplantation of fertilized eggs are necessary. It has been reported that offsprings can be limited to females by mating a male in which a Cas9 gene is knocked in to an X chromosome and a female in which three types of gRNA targeting Top1 which is a gene essential for generation are knocked in onto an autosomal chromosome (NPL 9: Yosef et al., 2019). A method has been reported in which the sex ratio of coming individuals is controlled by using parents having a CRISPR Cas9 system (Cas9 or gRNA) due to genetic recombination (NPL 10: C. Douglas et al., Nat. Commun 2021, doi.org/10.1038/s41467 021 27227 2). However, offsprings coming through such a method may be inappropriate to application to livestock animals because they have a foreign gene.

### PRIOR ART DOCUMENTS NON PATENT DOCUMENTS

[NPL 1] Alfieri et al., Journal of Cell Science, 2003, 116(11), 2149-2155
[NPL 2] Johnson, Sexing Mammalian Sperm for Production of Offspring: the State-of-the-art, Animal Reproduction Science 2000, 60-61, 93-107
[NPL 3] Lee et al., Theriogenology, 2004, 62, 1452-1458
[NPL 4] Veerhuis et al., Veterinary Immunology and Immunopathology, 1994, 42, 317-330
[NPL 5] Bredbacka, Reproduction Nutrition Development, 1998, 38, 605-613
[NPL 6] Harper et al., Human Reproduction, 1994, 9, 721-724
[NPL 7] HuaBin et al., Journal of Animal and Veterinary Advances 2012, 11, 1847-1852
[NPL 8] Zhao et al., Scientific Reports, 2019, 9(1), 1-9.
[NPL 9] Yosef et al., EMBO Reports, 2019, 20(8), 1-5
[NPL 10] C. Douglas et al., Nat. Commun., 2021, 12, 6926

### SUMMARY OF INVENTION

### Problem to be Solved by the Invention

An object of the present invention is to provide a method which enables sex selection in a non-human animal, and enables a coming individual to have no recombination gene, and a transgenic non-human animal giving birth to individuals of only one sex, which is used in the method.

### Means for Solving the Problem

In view of these circumstances, the present inventors have conducted intensive studies, and resultantly found that about a half of fertilized eggs obtained by mating a male non-human individual, in which an exogenous embryonic death induction gene is introduced onto an X chromosome or a Y chromosome and the embryonic death induction gene is expressed in the presence of a specific drug, with a female of the same species become fertilized eggs having an embryonic death induction gene, and treatment of the fertilized eggs in the presence of a specific drug causes cell death, leading to death of the fertilized eggs. As a result, among the chromosomes of the male, only fertilized eggs having chromosomes free of the embryonic death induction gene survive. Therefore, by this method, the sex of coming individuals can be controlled. More noteworthy is that coming individuals have no recombination gene, and therefore, when they are applied to experimental animals or livestock animals, it is possible to produce sex-selected individuals which are not influenced at all by a foreign gene. Specifically, the gist of the present invention is as follows.

[1] A male transgenic non-human animal in which one of an X chromosome and a Y chromosome comprises an exogenous embryonic death induction gene operatively connected to a drug-responsive promoter.

[2] The male transgenic non-human animal according to [1], wherein the drug-responsive promoter is selected from the group consisting of a tetracycline-responsive promoter, a cumate-responsive promoter, a hormone-responsive promoter and an RSL1-responsive promoter.

[3] A method for acquiring a pregnant non-human animal, comprising the step of mating a male transgenic non-human animal in which an X chromosome or a Y chromosome comprises an exogenous embryonic death induction gene operatively connected to a drug-responsive promoter and a female non-human animal of the same species which is free of an exogenous embryonic death induction gene.

[4] A method for selecting a fertilized egg free of an exogenous embryonic death induction gene, comprising the step of treating a fertilized egg of the pregnant non-human animal acquired by the method according to [3] with a drug to which the promoter responds.

[5] The method for selecting a fertilized egg according to [4], wherein the step of treatment with a drug is a step of administering the drug to the pregnant non-human animal, or a step of collecting a fertilized egg from the pregnant non-human animal and culturing the fertilized egg in a medium containing the drug.

[6] A method for producing a male non-human animal, comprising the steps of:
(A1) mating a male transgenic non-human animal in which an X chromosome comprises an exogenous embryonic death induction gene operatively connected to a drug-responsive promoter and a female non-human animal of the same species which is free of an exogenous embryonic death induction gene; and
(B1) treating a fertilized egg of a pregnant non-human animal obtained by the step (A1) with the drug.

[7] The method for producing a male non-human animal according to [6], wherein the step (B1) is a step of administering the drug to the pregnant non-human animal obtained by the step (A1), or a step of collecting a fertilized egg from the pregnant non-human animal obtained by the step (A1), and culturing the fertilized egg in a medium containing the drug.

[8] A method for producing a female non-human animal, comprising the steps of:
(A2) mating a male transgenic non-human animal in which a Y chromosome comprises an exogenous embryonic death induction gene operatively connected to a drug-responsive promoter and a female non-human animal of the same species which is free of an exogenous embryonic death induction gene; and
(B2) treating a fertilized egg of a pregnant non-human animal obtained by the step (A2) with the drug.

[9] The method for producing a female non-human animal according to [8],
wherein the step (B2) is a step of administering the drug to the pregnant non-human animal obtained by the step (A2), or a step of collecting a fertilized egg from the pregnant non-human animal obtained by the step (A2), and culturing the fertilized egg in a medium containing the drug.

[10] A method for producing a male non-human animal, comprising the steps of:
(A3) performing in vitro fertilization using sperm of a male transgenic non-human animal in which an X chromosome comprises an exogenous embryonic death induction gene operatively connected to a drug-responsive promoter and egg mother cells of a female non-human animal of the same species which is free of an exogenous embryonic death induction gene;
(B3) treating a fertilized egg obtained by the step (A3) with the drug; and
(C3) implanting a fertilized egg having survived the step (B3) in a foster mother.

[11] A method for producing a female non-human animal, comprising the steps of:
(A4) performing in vitro fertilization using sperm of a male transgenic non-human animal in which a Y chromosome comprises an exogenous embryonic death induction gene operatively connected to a drug-responsive promoter and egg mother cells of a female non-human animal of the same species which is free of an exogenous embryonic death induction gene;
(B4) treating a fertilized egg obtained by the step (A4) with the drug; and
(C4) implanting a fertilized egg having survived the step (B4) in a foster mother.

[12] An embryonic death induction gene expression system comprising: a first expression cassette comprising a drug-responsive promoter sequence, and an embryonic death induction gene arranged downstream of the promoter sequence; and a second expression cassette comprising a promoter sequence functioning in a fertilized egg, and a sequence encoding a drug-controlled trans-activator arranged downstream of the promoter sequence.

[13] The embryonic death induction gene expression system according to [12], wherein the first expression cassette is held in a first vector, and the second expression cassette is held in a second vector.

[14] The embryonic death induction gene expression system according to [12], wherein the first expression cassette and the second expression cassette are held in one vector.

[15] A method for producing a male transgenic non-human animal for sex selection, comprising introducing the embryonic death induction gene expression system according to any one of [12] to [14] into a fertilized egg, culturing an embryo, and then transplanting the embryo into a uterus of a pseudopregnant non-human animal to obtain an offspring.

### Effects of the Invention

According to the present invention, it is possible to control the sex of coming individuals in a non-human animal. Fertilized eggs fertilized with a sex chromosome having an exogenous embryonic death induction gene before implantation die, and an accordingly larger number of fertilized eggs fertilized with the other chromosome are implanted, so that it is possible to efficiently obtain offsprings of a required sex. More noteworthy is that coming individuals have no recombination gene, and therefore, when they are applied to experimental animals or livestock animals, it is possible to produce sex-selected individuals which are not influenced at all by a foreign gene.

### BRIEF DESCRIPTION OF THE DRAWING

FIG. 1 shows microscope photographs (Bright and GFP) of fertilized eggs with Tet-on-Bax-IRES-mem-AcGFP and fertilized eggs as a control (NT);

FIG. 2 shows microscope photographs of fertilized eggs with Tet-on-Bax-IRES-mem-AcGFP and fertilized eggs as a control (NT) after the fertilized eggs are cultured in Dox (+) medium or Dox (−) medium for 24 hours;

FIG. 3 schematically shows a knock-in region of Tet-on-Bax;

FIG. 4 shows the results of genotyping of fertilized eggs;

FIG. 5 shows microscope photographs (Bright and GFP) of 13 fertilized eggs into which pBS-Hprt-arm-CAG-EGFP and Cas9 RNP are co-injected;

FIG. 6 shows the results of genotyping of one fertilized egg in which GFP fluorescence is observed, among the fertilized eggs into which pBS-Hprt-arm-CAG-EGFP and Cas9 RNP are co-injected;

FIG. 7 shows the results of genotyping of genetically recombinant male mice having Tet-on-Bax on an X chromosome;

FIG. 8 shows a sex ratio of offsprings obtained by mating genetically recombinant male mice having Tet-on-Bax on an X chromosome and wild-type female mice;

FIG. 9 shows a sex ratio of offsprings obtained by mating genetically recombinant male mice having Tet-on-Bax on an X chromosome and wild-type female mice;

FIG. 10 schematically shows intragenic regions of exon 1 and exon 2 in a Ddx3y gene, which are selected as knock-in regions of Tet-on-Bax;

FIG. 11 shows microscope photographs (Bright and GFP) of 10 fertilized eggs in which GFP fluorescence is observed, among 22 fertilized eggs into which pBS-Dby-arm-CAG-EGFP and Cas9 RNP are co-injected;

FIG. 12 shows the results of genotyping of 22 fertilized eggs into which pBS-Dby-arm-CAG-EGFP and Cas9 RNP are co-injected;

FIG. 13 shows the results of genotyping of genetically recombinant male mice having Tet-on-Bax on a Y chromosome; and

FIG. 14 is a schematic diagram of the action of sex selection mice.

### DETAILED DESCRIPTION OF THE INVENTION

Hereinafter, the present invention will be described in detail. In the present specification, molecular biological techniques can be carried out by methods described in general experimental textbooks known to those skilled in the art, or similar methods unless otherwise specified. Terms used in the present specification have meanings that are commonly interpreted in the art unless otherwise stated.

According to the present invention, it is possible to provide a male transgenic non-human animal which can be used for sex selection and in which one of an X chromosome and a Y chromosome comprises an exogenous embryonic death induction gene operatively connected to a drug-responsive promoter. There are also provided a method for producing the male transgenic non-human animal, a method for acquiring a pregnant non-human animal by mating the male transgenic non-human animal and a female of the same species, a method for selecting a fertilized egg free of an exogenous embryonic death induction gene, a method for producing a male or female non-human animal using the foregoing methods, an embryonic death induction gene expression system for use in the foregoing methods, and the like.

### <Male Transgenic Non-Human Animal>

The present invention includes a male transgenic non-human animal in which one of an X chromosome and a Y chromosome comprises an exogenous embryonic death induction gene operatively connected to a drug-responsive promoter. The male transgenic non-human animal of the present invention is suitably used for sex selection. Specifically, about a half of fertilized eggs obtained by mating a male non-human individual, which comprises an exogenous embryonic death induction gene on an X chromosome or a Y chromosome and in which the embryonic death induction gene is expressed in the presence of a specific drug, with a female of the same species become fertilized eggs having an embryonic death induction gene, and treatment of the fertilized eggs in the presence of a specific drug causes cell death, leading to death of the fertilized eggs. Among chromosomes of the male transgenic non-human animal, only fertilized eggs having a chromosome free of the embryonic death induction gene survive. Therefore, the use of the male transgenic non-human animal enables control of the sex of coming individuals. A further advantage is that coming individuals have no recombination gene, and therefore, when they are applied to experimental animals or livestock animals, it is possible to produce sex-selected individuals which are not influenced at all by a foreign gene. A construct comprising the exogenous embryonic death induction gene operatively connected to a drug-responsive promoter (a construct comprising a drug-responsive promoter sequence, and an embryonic death induction gene arranged downstream of the promoter sequence) may be called a first expression cassette.

In the present invention, the drug-responsive promoter is not limited as long as it is a promoter that specifically respond to a specific drug, and examples thereof include tetracycline-responsive promoters, cumate-responsive promoters, hormone-responsive promoters, and RSL1-responsive promoters. Of these, tetracycline-responsive promoters and cumate-responsive promoters are preferable, and tetracycline-responsive promoters are more preferable, because of the high specificity and the low toxicity of the expression inducing substance.

When the tetracycline-responsive promoter is used in the present invention, one of an X chromosome and a Y chromosome of the male transgenic non-human animal of the present invention comprises a tet operator sequence (tetO), a tetracycline-responsive promoter sequence that is under control of the tet operator sequence, and an embryonic death induction gene arranged downstream of the promoter sequence.

The tet operator sequence (tetO) is a sequence to which a reverse tetracycline-controlled trans-activator (rtTA) can bind. Binding of rtTA activates the promoter that is under control of tetO. The sequence of tetO is not limited as long as it is a sequence having these characteristics.

The promoter sequence is functionally connected to tetO. Normally, the promoter sequence is connected to the downstream side (3′ side) of tetO. The distance between tetO and the promoter sequence is not limited as long as the promoter sequence is under control of tetO. Typically, the promoter sequence is arranged immediately after tetO (with no space therebetween). As the promoter sequence, a minimum promoter sequence is typically used. The term “minimum promoter sequence” refers to a sub-promoter sequence which defines a transcription start site, but cannot start efficient transcription by itself. The activation of the minimum promoter sequence depends on binding of rtTA controlled by a tetracycline-based compound to tetO. Specific examples of the minimum promoter sequence include promoters derived from a human cytomegalovirus (CMV).

Transcription is started from a TATA box in the minimum promoter. Normally, intron is present upstream of an intended gene for enhancing the stability of a transcription product. Specific examples of the intron sequence include rabbit-derived beta-globin intron.

Downstream of the exogenous embryonic death induction gene, a poly A addition signal sequence is arranged. The transcription is terminated by use of the poly A addition signal sequence. As the poly A addition signal sequence, a poly A addition sequence of the intended gene itself, a poly A addition sequence of SV40, a poly A addition sequence of a bovine-derived growth hormone gene, and the like can be used.

A gene for detection (a reporter gene, a cell- or tissue-specific gene, a selection marker gene or the like), an enhancer sequence, a WRPE sequence and the like may be included in the first expression cassette. The gene for detection is used for determination of success or failure and efficiency in introduction of an expression cassette, detection of expression of an exogenous embryonic death induction gene or determination of expression efficiency, selection and sampling of cells in which an embryonic death induction gene is expressed, and the like. On the other hand, expression efficiency is improved by use of the enhancer sequence. As the gene for detection, genes such as a neo gene imparting resistance to neomycin, a npt gene or a nptII gene imparting resistance to kanamycin and the like, an hph gene imparting resistance to hygromycin, and a dhfr gene or the like imparting resistance to methotrexate (each of which is a marker gene), a luciferase gene, a β-glucuronidase (GUS) gene, and a gene of a fluorescence protein such as GFP or a modified product thereof (EGFP, d2EGFP or the like) (each of which is a reporter gene), and epidermal growth factor receptor (EGFR) gene lacking an intracellular domain can be used. The gene for detection is connected to the embryonic death induction gene through, for example, a bicistronic control sequence (for example, a ribosome internal recognition sequence (IRES)) or a sequence encoding a self-cleaving peptide. Examples of the self-cleaving peptide include, but are not limited to, a 2A peptide derived from a Thosea asigna virus (T2A). As the self-cleaving peptide, a 2A peptide derived from a foot-and-mouth disease virus (FMDV) (F2A), a 2A peptide derived from an equine rhinitis A virus (ERAV) (E2A), a 2A peptide derived from a porcine teschovirus (PTV-1) (P2A), and the like are known.

For controlling the expression of a drug-specific exogenous embryonic death induction gene, the male transgenic non-human animal of the present invention comprises, in one of an X chromosome and a Y chromosome, two characteristic expression cassettes including the first expression cassette. Specifically, the male transgenic non-human animal of the present invention comprises, in a one of an X chromosome and a Y chromosome, a first expression cassette comprising a drug-responsive promoter sequence and an embryonic death induction gene arranged downstream of the promoter sequence as described above, and a second expression cassette comprising a promoter sequence functioning in a fertilized egg, and a sequence encoding a drug-controlled trans-activator arranged downstream of the promoter sequence.

The second expression cassette comprises a promoter sequence functioning in a fertilized egg, and a sequence encoding a drug-controlled trans-activator arranged downstream of the promoter sequence.

The promoter is not limited as long as it is a promoter functioning in a fertilized egg, and examples of the promoter that can be used include constitutive promoters, and minimum promoters. Specific examples of the promoter include CMV promoters, CAG promoters, SV40ori, retroviral LTP, SRα, EF1α, β actin promoters, and nuclear factor of activated T cells (NFAT)-controlled promoters.

Preferred examples of the drug-controlled trans-activator include a reverse tetracycline-controlled trans-activator (rtTA) that can be controlled by a tetracycline-based compound.

The rtTA does not bind to tetO in the absence of a tetracycline-based compound, and binds to tetO in the presence of a tetracycline-based compound. The rtTA is a fused protein comprising a rtet repressor (rTetR) and a transcription activation domain. The rTetR has been discovered as a variant of TetR (Gossen M, et al., Science 1995; 268: 1766-1769). As the transcription activation domain, an acidic activation domain, a proline-rich transcription activation domain, a serine/threonine-rich transcription activation domain, a glutamine-rich activation domain, and the like are used. A herpes simplex virus virion protein 16 (VP16) which is an acidic activation domain is preferable as a transcription activation domain.

The sequence encoding rtTA is arranged under control of the promoter. That is, in the second expression cassette, the promoter sequence and the sequence encoding rtTA are functionally connected.

As is the case with the first expression cassette, a gene for detection (a reporter gene, a cell- or tissue-specific gene, a selection marker gene or the like), an enhancer sequence, a WRPE sequence and the like may be included in the second expression cassette.

Specific examples of the second expression cassette include those having a structure in which a constitutive promoter (for example, a CMV promoter), a sequence encoding rtTA, and a poly A addition signal sequence are arranged in the stated order from the upstream side (5′ side).

Specific examples of the use of the tetracycline-responsive promoter include Tet-On/Off Advanced expression induction systems, and a Tet-On system is preferable because it is desirable to enable expression of a corresponding gene in the presence of tetracycline. That is, the system expresses a fused protein of reverse tetR (rtetR) and VP16 (rtTA: reverse tetracycline-controlled trans-activator) simultaneously. When a tetracycline-based compound is present, rtTA can bind to a tet operator sequence to induce expression of an operatively connected exogenous embryonic death induction gene. The Tet-On system can be obtained from Clontech Laboratories, Inc., and used.

Examples of the tetracycline-based compound include tetracycline, doxycycline, deoxycycline, anhydrotetracycline, cyanotetracycline, and chlortetracycline. Of these, tetracycline and doxycycline are preferable, and doxycycline is more preferable, from the viewpoint of excellent responsiveness.

Commercial products such as cumate-responsive promoters (Q-mate System, Krackeler Scientific, Inc., National Research Council (NRC), and the like), hormone (estrogen)-responsive promoters (WO 2006/129735, and GenoStat Inductive Expression System, Upstate Cell Signaling Solutions Company), and RSL1-responsive promoters (RheoSwitch Mammal Inductive Expression System, New England Biolabs Inc.) can be suitably used.

In the present invention, the exogenous embryonic death induction gene is not limited as long as it is a gene that is expressed by artificial induction to cause embryonic death, and examples of the preferred gene include apoptosis-related genes such as Bax, caspase 3, caspase 8, caspase 9, Bcl-xo, Bcl-xS, Bad and Bik.

In the present invention, the animal species of the male transgenic non-human animal is not limited as long as it is an animal other than a human, and examples thereof include mammals such as mice, rats, hamsters, dogs, cats, bovines, pigs, horses, goats, and sheep.

### <Method for Producing Male Transgenic Non-Human Animal for Sex Selection>

The present invention also includes a method for producing the male transgenic non-human animal for use in sex selection. The male transgenic non-human animal for sex selection according to the present invention can be produced by introducing, into a fertilized egg, an embryonic death induction gene expression system which comprises a first expression cassette comprising a drug-responsive promoter sequence and an embryonic death induction gene arranged downstream of the promoter sequence, and a second expression cassette comprising a promoter sequence functioning in the fertilized egg and a sequence encoding a drug-controlled trans-activator arranged downstream of the promoter sequence, culturing an embryo, and then transplanting the embryo into a uterus of a pseudopregnant non-human animal (foster mother) to obtain an offspring.

### (Embryonic Death Induction Gene Expression System)

In the method of the present invention, the embryonic death induction gene expression system introduced into a fertilized egg comprises a first expression cassette comprising a drug-responsive promoter sequence and an embryonic death induction gene arranged downstream of the promoter sequence, and a second expression cassette comprising a promoter sequence functioning in the fertilized egg and a sequence encoding a drug-controlled trans-activator arranged downstream of the promoter sequence. The first expression cassette and the second expression cassette are loaded into a vector for introduction into the fertilized egg.

In the present specification, the term “vector” refers to a nucleic molecule capable of transporting a nucleic acid molecule inserted thereinto to the inside of a target fertilized egg, and its form, origin and the like are not limited. It is possible to use various types of vectors. In the present invention, as the vector, any of a viral vector and a non-viral vector can be used. In the case of the viral vector, which manipulates a phenomenon in which a virus is transmitted to cells, high gene introduction efficiency is obtained.

Examples of the viral vector include retroviral vectors, lentiviral vectors, adenoviral vectors, adeno-associated viral vectors, herpesviral vectors, and Sendai-viral vectors. In the case of retroviral vectors, lentiviral vectors and adeno-associated viral vectors, among the viral vectors mentioned above, an intended gene incorporated into the vector is incorporated into a host chromosome, stable and long-term expression can be expected. Each viral vector can be prepared by a reported method, or with a commercially available dedicated kit.

Examples of the non-viral vector include plasmid vectors, liposome vectors, positively charged liposome vectors, YAC vectors, and BAC vectors. The system of the present invention may be constructed using transposon.

In an aspect of the system, a first expression cassette and a second expression cassette are loaded into separate vectors. That is, a vector for the first expression cassette (first vector) and a vector for the second expression cassette (second vector) are prepared. In this aspect, the first vector and the second vector are introduced into a fertilized egg. The order of introduction is not limited. The vectors may be introduced in parallel, or one of the vectors may be introduced first. It is preferable to introduce the second vector first. When this order is adopted, a fertilized egg into which the second vector is appropriately introduced is selected, or concentrated or damped, followed by introduction of the first vector, so that the introduction rate of the system of the present invention can be improved.

In another aspect of the system of the present invention, a first expression cassette and a second expression cassette are loaded into one vector. That is, a vector holding both the first expression cassette and the second expression cassette is prepared. In this aspect, introduction of one vector into a fertilized egg enables the obtainment of a fertilized egg containing the system of the present invention. Being able to introduce the system of the present invention by the operation of introducing one vector (one introduction operation) is a major advantage in wide use of the present invention. In the vector of this aspect, for example, the first expression cassette comprising a tet operator sequence, a promoter sequence that is under control of the tet operator sequence, and a death induction gene arranged downstream of the promoter sequence, and the second expression cassette comprising a promoter sequence functioning in the fertilized egg, and rtTA arranged downstream of the promoter sequence are held. Currently, as described above, a vector loaded with elements necessary for the system of the present invention (rtTA is expressed in a forward direction and an intended gene is expressed in a backward direction) is commercially available (Tet-One™ System: Clontech Laboratories, Inc.). This can be used to construct the system of the aspect in which the first expression cassette and the second expression cassette are loaded into one vector.

The scope of the present invention also includes the above-described embryonic death induction gene expression system.

(Introduction of Above System into Fertilized Egg, Culture of Embryo and Transplantation)

A recombinant expression vector comprising the system (a first vector loaded with a first expression cassette and a second vector loaded with a second expression cassette are used in combination, or a vector loaded with a first expression cassette and a second expression cassette is used) is introduced into a fertilized egg.

The above-described fertilized egg may be a fertilized egg acquired by in vitro fertilizing an unfertilized egg obtained by performing, on a female non-human animal, control of the sexual cycle and controlled ovarian hyperstimulation by administration of a gonadotropic hormone for the purpose of obtaining a large number of early embryos from one individual, with sperm of a male non-human animal of the same species, or may be a fertilized egg acquired from a female mouse immediately after natural mating, or may be a fertilized egg acquired by administering a gonadotropic hormone to a female non-human animal to induce superovulation, followed by mating with a male non-human mammal animal. The use of a fertilized egg obtained by in vitro fertilization is preferable in that it is easy to control the correct time for introduction of the recombinant vector into the fertilized egg.

The method of controlled ovarian hyperstimulation in a female non-human animal is preferably a method in which by, for example, intraperitoneal injection, for example, a follicle-stimulating hormone (pregnant mare serum gonadotropin, commonly abbreviated as PMSG) is first administered, and a luteinizing hormone (human chorionic gonadotropin, commonly abbreviated as hCG). The preferred dose and dosing interval of the hormone vary depending on the type of non-human animal. For example, when the non-human animal is a mouse or the like, normally, a method is preferable in which a follicle-stimulating hormone is administered, and after about 48 hours, a luteinizing hormone is administered to immediately obtain an unfertilized egg.

The fertilized egg into which the recombinant expression vector comprising the system is introduced is required to be a one-celled fertilized egg. This is because in a two-or-more-cells fertilized egg, there are a plurality of cells, and it is difficult to incorporate a gene into the sex chromosomes of all the cells.

The recombinant expression vector comprising the system can be introduced into a fertilized egg by a conventional method. Specific examples include a method of microinjection into the cytoplasm of a fertilized egg, and a lipofection method. Of these, a microinjection method is preferable. The microinjection method can be carried out by, for example, a conventional method using a known apparatus such as a micromanipulator. The fertilized egg placed in a microdroplet of an embryo culturing medium is taken under suction with a holding pipette, and fixed, and using an injection pipette, a DNA solution targeting an X chromosome and a DNA solution targeting a Y chromosome are directly injected into a female pronucleus and a male pronucleus, respectively. The fertilized egg after introduction of DNA is cultured until the blastocyst stage in the embryo culturing medium under 5% carbon dioxide/95% air by a microdroplet culture method or the like, and then transplanted into a uterine tube or a uterus of an embryo reception pseudopregnant female non-human animal (foster mother). The embryo reception female non-human animal is only required to be of the same species as an animal from which the early embryo transplanted is derived. For example, when a mouse early embryo is transplanted, ICR female mice (preferably about 8 to about 10 weeks old), and the like are preferably used.

As a method for bringing an embryo reception female non-human animal into a pseudopregnant state, for example, a method is known in which the female non-human animals that are confirmed to have a vaginal plug when mated with vasectomized (ligated) male non-human mammal animals of the same species (for example, in the case of mice, ICR male mice (preferably about 2 months old or older)) are selected.

For culturing fertilized eggs using an embryo generation medium, appropriate wells are filled with the embryo generation medium, and the fertilized eggs are transferred to the wells, and cultured. Culture conditions (CO₂ concentration, 02 concentration, temperature and humidity conditions) are not limited, and can be appropriately set. The conditions can be set to, for example, 5% CO₂, 5% O₂, 90% N₂, 38.0 to 38.5° C. (preferably 38.5° C.) and humidity 100%.

Whether introduced DNA is incorporated in chromosomal DNA or not can be tested by, for example, carrying out Southern hybridization or a PCR method to screen chromosomal DNA separated and extracted from the tail of a male offspring.

### <Sex Selection Method>

According to the present invention, it is possible to control the sex of coming individuals in a non-human animal. In addition, coming individuals have no recombination gene, and therefore, when they are applied to experimental animals or livestock animals, it is possible to produce sex-selected individuals which are not influenced at all by a foreign gene. Further, fertilized eggs fertilized with a sex chromosome having an exogenous embryonic death induction gene before implantation die, and an accordingly larger number of fertilized eggs fertilized with the other chromosome are implanted, so that it is possible to efficiently obtain offsprings of a required sex.

An embodiment of the sex selection method of the present invention is a method comprising:
(1) a method for acquiring a pregnant non-human animal, comprising the step of mating a male transgenic non-human animal in which an X chromosome or a Y chromosome comprises an exogenous embryonic death induction gene operatively connected to a drug-responsive promoter and a female non-human animal of the same species which is free of an exogenous embryonic death induction gene; and
(2) a method for acquiring a fertilized egg free of an exogenous embryonic death induction gene, comprising the step of treating a fertilized egg of the pregnant non-human animal acquired by the method (1) with a drug to which the promoter responds.

### (Method for Acquiring Pregnant Non-Human Animal)

The present method comprises the step of mating a male transgenic non-human animal in which an X chromosome or a Y chromosome comprises an exogenous embryonic death induction gene operatively connected to a drug-responsive promoter and a female non-human animal of the same species which is free of an exogenous embryonic death induction gene. The male transgenic non-human animal in which an X chromosome or a Y chromosome comprises an exogenous embryonic death induction gene operatively connected to a drug-responsive promoter is as described in the section “Male transgenic non-human animal” above. By mating the male transgenic non-human animal and a female non-human animal of the same species which is free of an exogenous embryonic death induction gene, a fertilized egg comprising a combination of an X chromosome or a Y chromosome comprising an exogenous embryonic death induction gene and derived from the male transgenic non-human animal, and an X chromosome free of an exogenous embryonic death induction gene, which is derived from the female non-human animal of the same species is formed. When the male transgenic non-human animal comprises an exogenous embryonic death induction gene on the X chromosome, the fertilized egg obtained after the mating is (X, X) or (X, Y) (the underline indicates inclusion of an exogenous embryonic death induction gene). When the male transgenic non-human animal comprises an exogenous embryonic death induction gene on the Y chromosome, the fertilized egg obtained after the mating is (X, X) or (X, Y) (the underline indicates inclusion of an exogenous embryonic death induction gene).

### (Method for Selecting Fertilized Egg Free of Exogenous Embryonic Death Induction Gene)

The present method comprises the step of treating a fertilized egg of the pregnant non-human animal acquired by the method for acquiring a pregnant non-human animal, with a drug to which the promoter responds. The step of treatment with a drug is a step of administering the drug to the pregnant non-human animal, or a step of collecting a fertilized egg from the pregnant non-human animal, and culturing the fertilized egg in a medium containing the drug.

For the fertilized egg of the pregnant non-human animal acquired by the method for acquiring a pregnant non-human animal, when the male transgenic non-human animal comprises an exogenous embryonic death induction gene on the X chromosome, the fertilized egg obtained after the mating is (X, X) or (X, Y) (the underline indicates inclusion of an exogenous embryonic death induction gene). When the male transgenic non-human animal comprises an exogenous embryonic death induction gene on the Y chromosome, the fertilized egg obtained after the mating is (X, X) or (X, Y) (the underline indicates inclusion of an exogenous embryonic death induction gene). Since the exogenous embryonic death induction gene present on each chromosome is operatively connected to a drug-responsive promoter, treatment with a specific drug induces expression of the embryonic death induction gene, and fertilized eggs comprising the gene die. That is, when the male transgenic non-human animal comprises an exogenous embryonic death induction gene on the X chromosome, female offsprings are not obtained because of the fertilized eggs (X, X) and (X, Y) obtained after the mating, (X, X) dies, and only male offsprings of (X, Y) can be obtained. When the male transgenic non-human animal comprises an exogenous embryonic death induction gene on the Y chromosome, male offsprings are not obtained because of the fertilized eggs (X, X) or (X, Y) obtained after the mating, (X, Y) dies, and only female offsprings of (X, X) can be obtained.

When the step of treatment with a drug is a step of administering the drug to the pregnant non-human animal, the pregnant non-human animal is made to orally ingest water, food or the like containing the drug, or given a preparation containing the drug to the pregnant non-human animal through intraperitoneal administration, subcutaneous administration, intravenous administration, intramuscular administration, sublingual administration, rectal administration, transvaginal administration or the like, whereby among the fertilized eggs of the pregnant non-human animal, fertilized eggs comprising the exogenous embryonic death induction gene can be led to death to select only fertilized eggs free of the exogenous embryonic death induction gene. From the pregnant non-human animal given the drug through any of the above-described methods, only offsprings derived from fertilized eggs free of the exogenous embryonic death induction gene are obtained. The concentration and the amount of the drug incorporated into water, food, a preparation or the like may be appropriately adjusted according to the type, the body weight, the age and the like of the pregnant non-human animal.

When the step of treating the drug is a step of collecting a fertilized egg from the pregnant non-human animal, and culturing the fertilized egg in a medium containing the drug, the peritoneal cavity of the pregnant non-human animal is opened, and fertilized eggs are taken out from the uterine tube, and cultured under 5% carbon dioxide/95% air in an embryo culturing medium (for example, M16 medium, modified Whitten medium, BWW medium, M2 medium, WM-HEPES medium, or BWW-HEPES medium) containing the drug. In the culture process, fertilized eggs comprising the exogenous embryonic death induction gene can be led to death to select only fertilized eggs free of the exogenous embryonic death induction gene.

The selected fertilized eggs are cultured in the embryo culturing medium under 5% carbon dioxide/95% air by a microdroplet culture method or the like, and then transplanted into the uterine tube or the uterus of a pseudopregnant embryo reception female non-human animal (foster mother). The method for bringing the embryo reception female non-human animal into a pseudopregnant state is as described above. The embryo reception female non-human animal is only required to be of the same species as an animal from which the early embryo transplanted is derived. For example, when a mouse early embryo is transplanted, ICR female mice (preferably about 8 to about 10 weeks old), and the like are preferably used. The sex of the resulting offspring can be tested by carrying out Southern hybridization or a PCR method to screen chromosomal DNA separated and extracted from the tail of an offspring.

Another embodiment of the sex selection method of the present invention is a method comprising:
(1′) a method for acquiring a fertilized egg, comprising the step of performing in vitro fertilization using sperm of a male transgenic non-human animal in which an X chromosome or a Y chromosome comprises an exogenous embryonic death induction gene operatively connected to a drug-responsive promoter and egg mother cells of a female non-human animal of the same species which is free of an exogenous embryonic death induction gene;
(2′) a method for selecting a fertilized egg free of an exogenous embryonic death induction gene, comprising the step of treating the fertilized egg acquired by the method (1′) with a drug to which the promoter responds; and
(3′) a method of implanting a fertilized egg having survived the method (2′) in a foster mother.

### (Method for Acquiring Fertilized Egg)

The present method comprises the step of performing in vitro fertilization using sperm of a male transgenic non-human animal in which an X chromosome or a Y chromosome comprises an exogenous embryonic death induction gene operatively connected to a drug-responsive promoter and egg mother cells of a female non-human animal of the same species which is free of an exogenous embryonic death induction gene. The male transgenic non-human animal in which an X chromosome or a Y chromosome comprises an exogenous embryonic death induction gene operatively connected to a drug-responsive promoter is as described in the section “Male transgenic non-human animal” above. By performing in vitro fertilization using sperm of the male transgenic non-human animal and egg mother cells of a female non-human animal of the same species which is free of an exogenous embryonic death induction gene, a fertilized egg comprising a combination of an X chromosome or a Y chromosome comprising an exogenous embryonic death induction gene and derived from the male transgenic non-human animal, and an X chromosome free of an exogenous embryonic death induction gene, which is derived from the female non-human animal of the same species is formed. When the male transgenic non-human animal comprises an exogenous embryonic death induction gene on the X chromosome, the fertilized egg obtained after in vitro fertilization is (X, X) or (X, Y) (the underline indicates inclusion of an exogenous embryonic death induction gene). When the male transgenic non-human animal comprises an exogenous embryonic death induction gene on the Y chromosome, the fertilized egg obtained after in vitro fertilization is (X, X) or (X, Y) (the underline indicates inclusion of an exogenous embryonic death induction gene).

The egg mother cells of the female non-human animal of the same species which is free of an exogenous embryonic death induction gene may be unfertilized eggs obtained by performing, on a female non-human animal, control of the sexual cycle and controlled ovarian hyperstimulation by administration of a gonadotropic hormone for the purpose of obtaining a large number of early embryos from one individual.

The method for inducing superovulation in a female non-human animal is preferably a method in which by, for example, intraperitoneal injection, for example, a follicle-stimulating hormone (pregnant mare serum gonadotropin, commonly abbreviated as PMSG) is first administered, and a luteinizing hormone (human chorionic gonadotropin, commonly abbreviated as hCG). The preferred dose and dosing interval of the hormone vary depending on the type of non-human animal. For example, when the non-human animal is a mouse or the like, normally, a method is preferable in which a follicle-stimulating hormone is administered, and after about 48 hours, a luteinizing hormone is administered to immediately obtain an unfertilized egg.

### (Method for Selecting Fertilized Egg Free of Exogenous Embryonic Death Induction Gene)

The present method comprises the step of treating the fertilized egg acquired by the above-described method with a drug to which the promoter responds. The step of treatment with the drug is a step of culturing the fertilized egg in a medium containing the drug.

Examples of the medium containing the drug include embryo culturing media (for example, M16 medium, modified Whitten medium, BWW medium, M2 medium, WM-HEPES medium, or BWW-HEPES medium) containing the drug. The fertilized egg is cultured in the medium under 5% carbon dioxide/95% air. In the culture process, fertilized eggs comprising the exogenous embryonic death induction gene can be led to death to select only fertilized eggs free of the exogenous embryonic death induction gene.

### (Method for Implanting Fertilized Egg in Foster Mother)

The selected fertilized eggs are cultured in the embryo culturing medium under 5% carbon dioxide/95% air by a microdroplet culture method or the like, and then transplanted into the uterine tube or the uterus of a pseudopregnant embryo reception female non-human animal (foster mother). The method for bringing the embryo reception female non-human animal into a pseudopregnant state is as described above. The embryo reception female non-human animal is only required to be of the same species as an animal from which the early embryo transplanted is derived. For example, when a mouse early embryo is transplanted, ICR female mice (preferably about 8 to about 10 weeks old), and the like are preferably used. The sex of the resulting offspring can be tested by carrying out Southern hybridization or a PCR method to screen chromosomal DNA separated and extracted from the tail of an offspring.

The sex selection method of the present invention comprises a method for producing a male non-human animal and a method for producing a female non-human animal, which are as follows.

### (Method for Producing Male Non-Human Animal)

An embodiment of the method for producing a male non-human animal according to the present invention is as follows:
a method for producing a male non-human animal, comprising the steps of:
(A1) mating a male transgenic non-human animal in which an X chromosome comprises an exogenous embryonic death induction gene operatively connected to a drug-responsive promoter, and a female non-human animal of the same species which is free of an exogenous embryonic death induction gene; and
(B1) treating a fertilized egg of a pregnant non-human animal obtained by the step (A1) with the drug.

Another embodiment of the method for producing a male non-human animal according to the present invention is as follows:
a method for producing a male non-human animal, comprising the steps of:
(A1′) performing in vitro fertilization using sperm of a male transgenic non-human animal in which an X chromosome comprises an exogenous embryonic death induction gene operatively connected to a drug-responsive promoter and egg mother cells of a female non-human animal of the same species which is free of an exogenous embryonic death induction gene;
(B1′) treating a fertilized egg obtained by the step (A1′) with the drug; and
(C1′) implanting a fertilized egg having survived the step (B1′) in a foster mother.

By this method, male non-human animals can be selectively acquired. For the specific descriptions of the steps, those in the embodiment of the sex selection method of the present invention can be applied.

### (Method for Producing Female Non-Human Animal)

An embodiment of the method for producing a female non-human animal according to the present invention is as follows:
a method for producing a female non-human animal, comprising the steps of:
(A2) mating a male transgenic non-human animal in which a Y chromosome comprises an exogenous embryonic death induction gene operatively connected to a drug-responsive promoter, and a female non-human animal of the same species which is free of an exogenous embryonic death induction gene; and
(B2) treating a fertilized egg of a pregnant non-human animal obtained by the step (A2) with the drug.

Another embodiment of the method for producing a female non-human animal according to the present invention is as follows:
a method for producing a female non-human animal, comprising the steps of:
(A2′) performing in vitro fertilization using sperm of a male transgenic non-human animal in which a Y chromosome comprises an exogenous embryonic death induction gene operatively connected to a drug-responsive promoter and egg mother cells of a female non-human animal of the same species which is free of an exogenous embryonic death induction gene;
(B2′) treating a fertilized egg obtained by the step (A2′) with the drug; and
(C2′) implanting a fertilized egg having survived the step (B2′) in a foster mother.

By this method, female non-human animals can be selectively acquired. For the specific descriptions of the steps, those in the embodiment of the sex selection method of the present invention can be applied.

### Example

The present disclosure will be described in detail in Examples below, but the present disclosure is not interpreted in a way limited by these Examples.

### 1. Suppression of Generation of Fertilized Eggs by Expression of Bax

Bax (Bcl-2-associated X protein) is a gene that is opposite in function to Bcl-2 (B-cell lymphoma 2) which is a gene that suppresses apoptosis. The present experiment was aimed at demonstrating that fertilized eggs are led to embryonic death by constructing a mechanism in which the fertilized eggs are forced to express Bax and that the sex of mammal animals can be selected by taking advantage of the apoptosis being induced in a sex chromosome-specific manner. In the present experiment, for testing whether expression of Bax induced apoptosis in fertilized eggs, fertilized eggs into which Tet-on-Bax had been randomly inserted was cultured in a Dox-containing medium to test the effect of expression of Bax. In the present experiment, experiments were conducted by a method which enables random integration with high efficiency using PiggyBac transposon in mouse fertilized eggs (Suzuki et al., 2015: Journal of Reproduction and Development, 61 (3), 241-244). Fluorescent labeling was performed by adding IRES-mem-AcGFP at the downstream of the Tet-on 3G protein gene that is constantly expressed in the Tet-on system, so that it was possible to calculate the introduction efficiency. Specific experimental methods and results are shown below.

### Materials and Methods

### 1) Superovulation Treatment

To 5- to 9-week-old ICR female mice raised in an artificially lit environment (from 9:00 in the morning to 23:00), 7.5 IU of a pregnant mare serum gonadotropin (eCG: equine chorionic gonadotropin, ASKA Animal Health Co., Ltd.) was intraperitoneally administered, and after about 48 hours, 7.5 IU of human chorionic gonadotropin (hCG, ASKA Animal Health Co., Ltd.) was intraperitoneally administered to perform controlled ovarian hyperstimulation.

### 2) In Vitro Fertilization and Culture of Embryo

About 14 hours after the injection of hCG, the mice were killed by cervical vertebra dislocation, the uterine tube was isolated, and unfertilized eggs were then collected, and transferred to 100 μl of a dropping medium prepared using in vitro fertilization medium HTF containing 4 mg/ml of BSA (A3311; SIGMA Chemical Co., St. Louis, MO) (Table 1). Sperm was extracted from the tail of epididymis of male mice of the same species, and pre-cultured in 100 μl of an in vitro fertilization medium for 1.5 hours. After the pre-culturing, the sperm was introduced into the dropping medium containing the unfertilized eggs such that the sperm concentration was 2×10⁵ cells/mi, and the unfertilized eggs were subjected to insemination for 2 hours to perform in vitro fertilization. The embryos fertilized in vitro were cultured under conditions of 5% CO₂ and 37° C. in KSOM medium containing 4 mg/mi of BSA and amino acids (Table 1).

TABLE 1

| | Amounts | | | | |
|---|---|---|---|---|---|
| Components | HTF | FHM | | KOSM | |
| NaCl | 593.8 mg/100 ml | 555.2 | mg/100 ml | 555.2 | mg/100 ml |
| KCl | 35.0 mg/100 ml | 18.6 | mg/100 ml | 18.6 | mg/100 ml |
| CaCl₂ | 57.0 mg/100 ml | 19.0 | mg/100 ml | 19.0 | mg/100 ml |
| KH₂PO₄ | 5.4 mg/100 ml | 4.8 | mg/100 ml | 4.8 | mg/100 ml |
| MgSo₄•7H₂O | 4.9 mg/100 ml | 4.9 | mg/100 ml | 4.9 | mg/100 ml |
| NaHCO₃ | 210.0 mg/100 ml | 33.6 | mg/100 ml | 210.0 | mg/100 ml |
| Glucose | 50.0 mg/100 ml | 3.6 | mg/100 ml | 3.6 | mg/100 ml |
| HEPES | — | 238.3 | mg/100 ml | — | |
| (Acid) | | | | | |
| HEPES | — | 260.3 | mg/100 ml | — | |
| (Na salt) | | | | | |
| L-Glutamine | — | 14.6 | mg/100 ml | 14.6 | mg/100 ml |
| Na-pyruvate | 3.7 mg/100 ml | 2.2 | mg/100 ml | 2.2 | mg/100 ml |
| Na-Lactate | 0.34 ml/100 ml | 0.1852 | ml/100 ml | 0.1852 | ml/100 ml |
| (60% syrup) | | | | | |
| Streptomycin | 5.0 mg/100 ml | 5.0 | mg/100 ml | 5.0 | mg/100 ml |
| Penicillin G | 6.3 mg/100 ml | 6.3 | mg/100 ml | 6.3 | mg/100 ml |
| EDTA•2Na | — | 100.0 | μl/100 ml | 100.0 | μl/100 ml |
| (110 mM | | | | | |
| solution) | | | | | |
| BSA | 4.0 mg/ml | 1.0 | mg/100 ml | 1.0 | mg/ml |

| | | | | |
|---|---|---|---|---|
| EAA(MEM) | — | — | 1.0 | ml/100 ml |
| NEAA | — | — | 500.0 | μl/100 ml |

### 3) Construction of Vector

A protein translation region (coding sequence: CDS) of Bax was loaded into a pBlueScript Sk(−) plasmid (Takara Bio Inc.). Here, using KOD-plus (TOYOBO CO., LTD.), nested PCR was performed in two stages, one of which corresponded to an outer primer located at both sides of a DNA region containing an intended amplicon and the other of which corresponded to a nested primer amplifying an intended target region, so that CDS of Bax was amplified from total cDNA of the ovary of the mice. A FLAG tag (DYKDDDDK) and a subsequent linker sequence were inserted at the downstream of the T7 promoter of the pBlueScript Sk(−) plasmid, and restriction enzyme treatment was then performed using Not I (New England Biolabs) and Xba I (New England Biolabs). A PCR product on which restriction enzyme treatment had been similarly performed was inserted into a vector plasmid by Ligation high (TOYOBO CO., LTD.).

Next, pTet-OneVector contained in Tet-One Inducible Expression System (Takara Bio Inc.), and the Bax gene were each amplified by performing PCR with KOD-plus neo (TOYOBO CO., LTD.), and pTet-on-Bax was prepared using Gibson Assembly Master Mix (New England Biolabs).

Next, IRES-mem-AcGFP was obtained using a pAcGFP-mem vector (Clonetech Laboratories, Inc.) and a fragment of the IRES sequence. The pTet-on-Bax and IRES-mem-AcGFP were amplified using a primer shown in the table, and IRES-mem-AcGFP was inserted at the downstream of the Tet-on 3G gene to obtain pTet-on-Bax-IRES-mem-AcGFP.

Next, a region other than CAG-TagRFP which includes inverted Terminal Repeats (ITR) of pPB-CAG-TagRFP, and the gene cassette of Tet-on-Bax-IRES-mem-AcGFP were each amplified by performing PCR with KOD-plus neo, and the fragments were bound with Gibson assembly Master Mix to obtain pPB-Tet-on-Bax-IRES-AcGFP. Table 2 shows the primers used.

TABLE 2

| SEQ. | | |
|---|---|---|
| No. | names of primers | sequences |
| 1 | Bax OF | GCACGTCCACGATCAGTCA |
| 2 | Bax OR | CCTTTCCCCTTCCCCCATTC |
| 3 | Bax CDS NotI | AAATCTAGAGGTGGTGGT |
| | and XbaI f | GGTTCCGGTGACGGGTCC |
| | | GGGGAGGAGCT |
| 4 | Bax CDS NotI | TTTGCGGCCGCTCAGCCCA |
| | and XbaI r | TCTTCTTCCAGA |
| 5 | pTetOne to | GAATTCTTTACGAGGGTAG |
| | GOI f | GAAGTG |
| 6 | pTetOne to | GGATCCAGATCTCTGCAG |
| | GENE r | CATATG |
| 7 | Bax to | GCAGAGATCTGGATCCTCAG |
| | pTetOne f | CCCATCTTCTTCCAGATGG |
| 8 | Bax to | CCCTCGTAAAGAATTCATGG |
| | pTetOne r | ACGGGTCCGGGGAGCAG |
| 9 | IRES-GFP to | GTAAATCTCCAGAGGCCGTTTT |
| | TetBax f | GTGGAACTACTATAACTCGAG |
| 10 | IRES-GFP to | ATCCCCTGATTCTGTCATGCCT |
| | pTetbax r | GCTATTGTCTTCCCAATC |
| 11 | TetBax to | ACAGAATCAGGGGATAACGCAGG |
| | IRES-GFP f | |
| 12 | TetBax to | CCTCTGGAGATTTACCCGGGGAG |
| | IRES-GFP r | |
| 13 | TetBax-IRES- | ATAGATATCGTCGACTCCGCGC |
| | GFP to pPB f | ACATTTCCCCGAAAAG |
| 14 | TetBax-IRES- | AGTAACAAAGGATCCCGAGGGA |
| | GFP to pPB r | GCTTCCAGGGGGAAAC |
| 15 | pPB to Gene f | GGATCCTTTGTTACTTTATAGAAG |
| 16 | pPB to Gene r | ATGTCGACGATATCTATAACAAG |

4) Microinjection of Plasmid and hyPBase mRNA

About 3 to 5 pl of a mixture of 30 ng/μl of pPB-Tet-on-Bax-IRES-AcGFP and 30 ng/μl of hyPBase mRNA was microinjected into the cytoplasm of fertilized eggs 3 to 4 hours after fertilization. A treatment section where 30 ng/μl of pPB-CAG-TagRFP and 30 ng/μl of hyPBase had been similarly microinjected was also prepared as a control. Further, for examining influences of Dox on the fertilized egg, a fertilized egg which had not undergone microinjection was also prepared.

### 5) Observation

Fluorescence of fertilized eggs was observed 96 hpi, and the ratio of occurrence of random integration was calculated. A half of the fertilized eggs of each treatment section were observed in KISOM medium containing Dox at 100 ng/ml, and others were observed in common KISOM medium for 24 hours.

### (Results and Discussions)

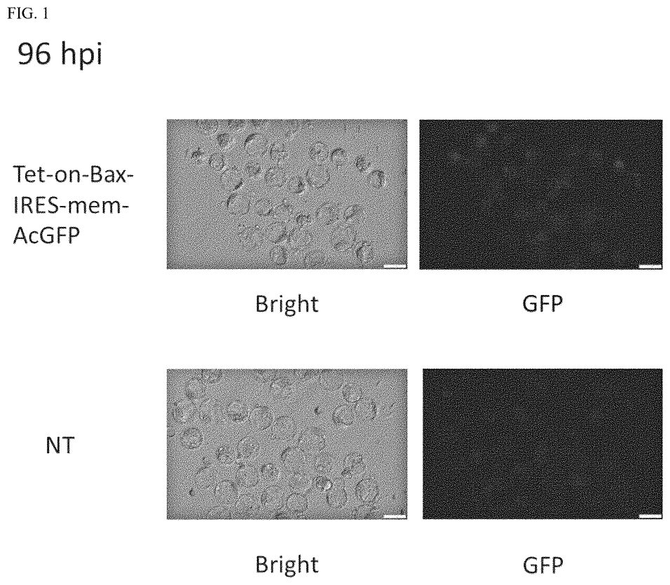

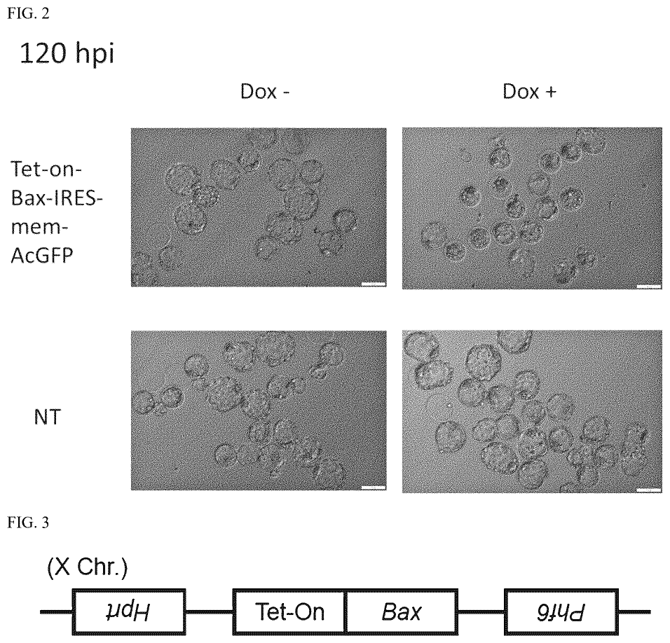

Fluorescence of fertilized eggs into which Tet-on-Bax-IRES-mem-AcGFP had been inserted was observed, and the results showed that fluorescence was observed for 26 out of 50 fertilized eggs. The results are shown in FIG. 1. Subsequently, fertilized eggs into which Tet-on-Bax-IRES-mem-AcGFP had been inserted were cultured in a Dox-containing medium, and 13 fertilized eggs out of 24 fertilized eggs (54%) underwent apoptosis. The results are shown in FIG. 2. On the other hand, all fertilized eggs were normally generated in the treatment section where fertilized eggs into which Tet-on-Bax-IRES-mem-AcGFP had been inserted were cultured in common KSOM, and the treatment section of NT. Table 3 collectively shows the results.

TABLE 3

| | Dox− | Dox+ | Dox− | Dox+ |
|---|---|---|---|---|
| | Tet- | Tet- | No | No |
| | on-Bax | on-Bax | Treatment | Treatment |
| Number of live embryos | 21 | 11 | 19 | 20 |
| Number of dead embryos | 0 | 13 | 0 | 0 |
| Number of total embryos | 21 | 24 | 19 | 20 |
| Percentage of dead embryos | 0 | 54 | 0 | 0 |

When fertilized eggs into which Tet-on-Bax-IRES-mem-AcGFP had been inserted were cultured in a Dox-containing medium, 54% of the fertilized eggs underwent apoptosis. This percentage is consistent with the percentage of fertilized eggs for which fluorescence was observed. This showed that it was possible to induce apoptosis with a high probability by adding Dox to fertilized eggs having Tet-on-Bax on the genome. Cell death was not observed for the treatment section where fertilized eggs into which Tet-on-Bax had been inserted were cultured in a Dox-free medium, and thus, it was revealed that a lethal amount of Bax was not expressed in the absence of Dox. Further, in culturing of the fertilized eggs of No-treatment in a Dox-containing medium, cell death was not observed in any of the treatment sections, and thus, it was revealed that fertilized eggs having no Tet-on-Bax were not led to embryonic death in a Dox-containing medium at 100 ng/μl. These results demonstrated that it was possible to select the sex by knocking in Tet-on-Bax to a sex chromosome.

### 2. Preparation of Genetically Recombinant Mice Having Tet-On-Bax on X Chromosome

The present experiment was aimed at preparing genetically recombinant male mice having Tet-on-Bax on an X chromosome. With reference to a prior study in which mCherry or eGFP is knocked in to an X chromosome (Kobayashi et al., 2016: Development (Cambridge), 143(16), 2958-2964), the intragenic regions of a Hprt gene and a Phf6 gene (hereinafter, described as Hprt) were selected as knock-in regions (FIG. 3).

To date, there have been a large number of reports on the technique of knocking in a gene cassette on a mega-base scale. In the present study, a technique was adopted in which a plasmid having Cas9 RNP (Cas9 ribonucleoprotein) with Cas9 protein and gRNA bound to each other and a homology arm at both ends of a gene cassette is microinjected into the pronucleus of a mouse fertilized egg. This technique has two advantages. The first is high knock-in efficiency. In a study intended to knock in an EGFP gene with a Rosa26 locus as a knock-in region, knock-in was confirmed for 70% of fertilized eggs. The second is the low frequency of non-specific insertion. It is known that adoption of plasmid DNA as donor DNA reduces the frequency of non-specific insertion. In the present study, sex selection cannot be appropriately performed if Tet-on-Bax is non-specifically inserted, and therefore, being able to reduce the non-specific insertion frequence is a major advantage. In the present experiment, first, fertilized eggs in which only Cas9 RNP had been microinjected into the cytoplasm were used as a sample, and sequencing analysis was performed on regions containing gRNA, thereby confirming cleavage efficiency. Next, a GFP gene was knocked in to Hprt to confirm the knock-in efficiency. Further, genetically recombinant mice were prepared in which Tet-on-Bax-IRES-AcGFP was knocked in to Hprt. Specific descriptions are given below.

2-1. Confirmation of Cleavage Efficiency of Hprt gRNA

In the present experiment, Cas9 RNP was microinjected into fertilized eggs to induce knock-out of a target sequence, the fertilized eggs were cultured 96 hpi, and Sanger sequencing analysis was then performed on regions containing the target sequence of gRNA, thereby confirming that knock-out occurred with a high probability. Specific experimental methods and results are shown below.

### Materials and Methods

1) Preparation of gRNA

Using CrisprDirect, gRNA was designed in intragenic regions of a Hprt gene and a Phf6 gene. Table 4 shows the sequence of gRNA. The gRNA was prepared by mixing 18 μM of crRNA (IDT) and 18 μl of tracrRNA (IDT) using Nuclease-free Duplex Buffer, followed by treatment at 95° C. for 15 minutes and at 25° C. for 5 minutes.

TABLE 4

| SEQ. | | |
|---|---|---|
| No. | gRNA | sequences |
| 17 | Hprt | GGCTAAAGAGTTGAACGCAAAGG |

### 2) Extraction of Egg Mother Cells, In Vitro Fertilization and Culture of Embryos

The same method as in the section “1. Suppression of generation of fertilized eggs by expression of Bax” was carried out.

### 3) Preparation of Cas9 RNP

Cas9 RNP was prepared by performing mixing in 5 μl of Opti-MEM (Life Technologies Corporation) containing 100 ng/μl of gRNA and 100 ng/μl of Cas9 Nuclease protein NLS (NIPPON GENE CO., LTD.).

4) Microinjection of Cas9 RNP into Cytoplasm

About 3 to 5 μl of the prepared Cas9 RNP prepared was microinjected into the cytoplasm of fertilized eggs 6 to 8 hours after insemination.

### 5) Genotyping of Fertilized Eggs

The fertilized eggs into which Cas9 RNP had been microinjected were cultured to blastocysts, then collected in 8 μl per fertilized egg of an embryo typing buffer (Table 5) (containing 200 μg/ml of proteinase K (Kanto Chemical Co., Inc.)), and treated at 55° C. for 30 minutes and at 95° C. for 10 minutes to extract genomes. With the extracted genome as a template, a region containing gRNA was amplified using KOD-FX neo (TOYOBO CO., LTD.). Column purification was performed on the PCR product, and Sanger sequencing analysis using primers shown in Table 6 was outsourced to Azenta Life Sciences.

TABLE 5

| Components | Amounts (mL) |
|---|---|
| Tris-HCl (1M, ph8.8) | 1.00 |
| KCl(5M) | 1.00 |
| NP-40 | 0.45 |
| Tween-20 | 0.45 |
| MgCl₂ (500 mM) | 0.50 |
| Total Volume | 100.00 |

TABLE 6

| SEQ. | | |
|---|---|---|
| No. | names of primers | sequences |
| 18 | Hprt KO check f | CCAAATGAACACAG |
| | | TCAAGGCTGAATC |
| 19 | Hprt KO check r | AGAATCAATGCTTC |
| | | CAGTGCTACCAAA |

### (Results and Discussions)

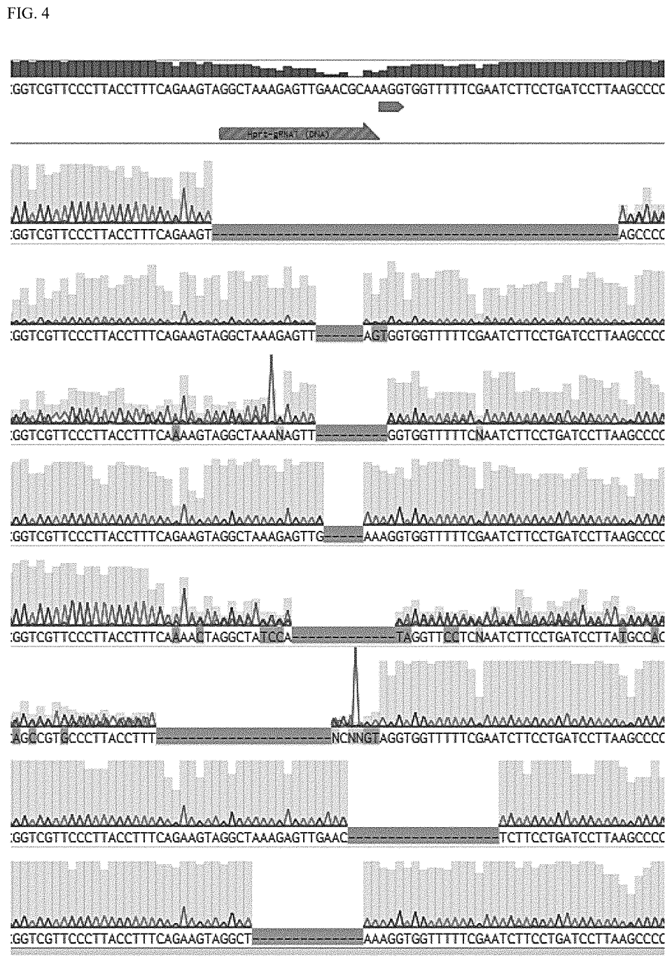

In all the 8 fertilized eggs analyzed by Sanger sequencing, deletion of a region complementary to gRNA was confirmed. The results are shown in FIG. 4. The gRNA sequence with high cleavage efficiency was used in subsequent experiments.

### 2-2. Knock-In of GFP Gene to Hprt Region

In the present experiment, a GFP gene was knocked in to a Hprt region to confirm the knock-in efficiency and the possibility of occurrence of non-specific insertion. Specific experimental methods and results are shown below.

### Materials and Methods

1) Construction of pBS-HprtArm-CAG-EGFP Vector

The intragenic regions of a Hprt gene and a Phf6 gene on an X chromosome were amplified from genomic DNA of the ovary of mice by performing PCR using KOD-plus-neo (TOYOBO CO., LTD.). The PCR product was inserted into the prepared pBS-SK(−)-FLAG vector using In-Fusion HD Cloning Kit, and then transformed into Escherichia coli DH5u to prepare a pBS-HprtArm vector. Next, the CAG-EGFP sequence was amplified from pR26 CAG/GFP Asc (Addgene) by performing PCR using KOD-plus-neo, followed by preparation of pBS-HprtArm-CAG-EGFP using Gibson Assembly Master Mix (NEW ENGLAND Biolabs). Table 7 shows the primer sequences.

TABLE 7

| SEQ. | names of | |
|---|---|---|
| No. | primers | sequences |
| 20 | Hprt-arm f | ggctttcaccgtagttcact |
| | | ggagact |
| 21 | Hprt-arm r | cacagtgactcttgtttcCC |
| | | TCTCCCA |
| 22 | CAG-EGFP | agggagctgcagtggagtaggc |
| | to vector f | |
| 23 | CAG-EGFP | gacaacgcccacacaccaggtt |
| | to vector r | |

### 2) Extraction of Egg Mother Cells, In Vitro Fertilization and Culture of Embryos

The same method as in the section “1. Suppression of generation of fertilized eggs by expression of Bax” was carried out.

### 3) Microinjection

Six to eight hours after fertilization, microinjection was performed on the female pronucleus for half of the fertilized eggs and on the male pronucleus for others.

### 4) Observation/Genotyping of Fertilized Eggs

Fluorescence of the fertilized eggs was observed 96 hpi. In the same manner as in “2-1”, “5)”, genotyping was performed on all the fertilized eggs.

### (Results and Discussions)

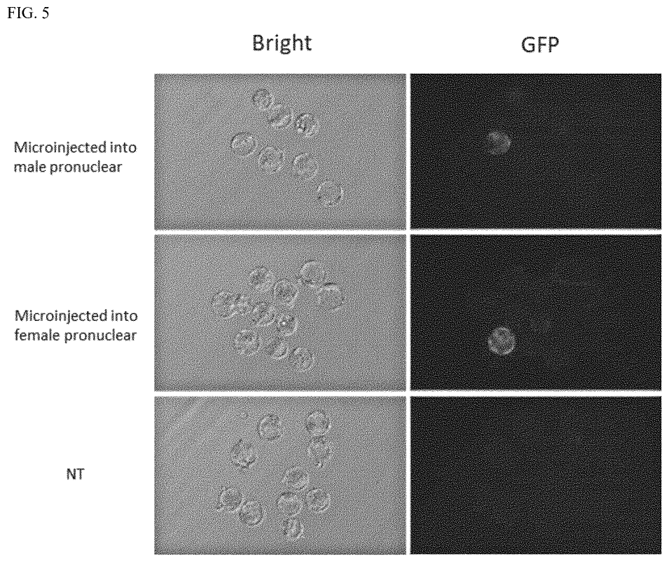

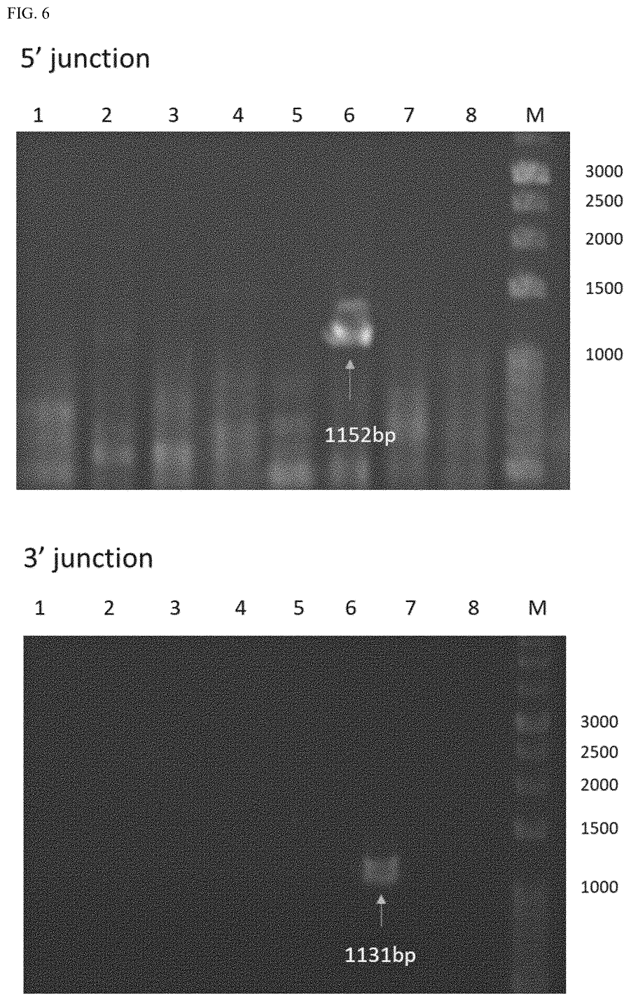

GFP fluorescence was observed for 2 out of the 13 fertilized eggs into which pBS-Hprt-arm-CAG-EGFP and Cas9 RNP had been co-injected. The results are shown in FIG. 5. It was confirmed by genotyping that in one of the two fertilized eggs, knock-in to an appropriate region had been performed. The results are shown in FIG. 6.

The knock-in efficiency was 7.7% when simply calculated. This time, DNA was microinjected into the male pronucleus for a half of the fertilized eggs. Therefore, if a half of the relevant fertilized eggs are male, the male pronuclei of these fertilized eggs have no X chromosome. Given the above, the knock-in efficiency may be further high. In one out of the two fertilized eggs where fluorescent was observed, a band with an appropriate size was not obtained. This indicates that non-specific insertion is considered to have occurred in the homologous recombination method adopted this time, and therefore, in future preparation of genetically recombinant mice in which Tet-on-Bax is knocked in, it is necessary to confirm that non-specific insertion does not occur.

### 2-3. Preparation of Genetically Recombinant Mice Having Tet-On-Bax on X Chromosome

In the present experiment, genetically recombinant male mice having Tet-on-Bax in the intragenic regions of the Hprt gene and the Phf6 gene of an X chromosome were prepared. Specific experimental methods and results are shown below.

### Materials and Methods

1) Preparation of pBS-HprtArm-Tet-on-Bax-IRES-AcGFP

Using, as a template, the pBS-HprtArm-CAG-EGFP prepared in accordance with the section “2-2. Knock-in of GFP gene to Hprt region”, a region excluding a CAG-EGFP cassette was amplified using KOD-plus neo (TOYOBO CO., LTD.), and the gene cassette region of the pTet-on-Bax-IRES-AcGFP prepared according to the section “1. Suppression of generation of fertilized eggs by expression of Bax” was amplified using KOD-plus neo. The two DNA fragments were bound with Gibson Assembly Master Mix (NEW ENGLAND BioLabs Inc.) to prepare pBS-HprtArm-Tet-on-Bax-IRES-AcGFP.

### 2) Collection of Egg Mother Cells, In Vitro Fertilization and Culture of Embryos

The same method as in the section “1. Suppression of generation of fertilized eggs by expression of Bax” was carried out.

### 3) Microinjection

The same microinjection method as in the section “2-2. Knock-in of GFP gene to Hprt region” was carried out. The microinjection was performed on the female pronucleus.

### 4) Transplantation of Embryos

After the microinjection, the fertilized eggs were cultured for 96 hours, and then transplanted into the uterus of pseudopregnant ICR mice using mNSET Device for Mice 60010 (ParaTechs). In the transplantation, the fertilized eggs of wild-type mice were mixed with the fertilized eggs in which microinjection had been performed, and the mixed fertilized eggs were transplanted at the same time such that the total number of transplants was 20.

### 5) Genotyping of Offsprings

The fingers of 1.5-week-old offsprings were cut, treated with Proteinase K for 24 hours, and then extracted with phenol to obtain genomic DNA. With the obtained genomic DNA as a template, PCR was performed using KOD-FX neo (TOYOBO CO., LTD.), followed by genotyping. Table 8 shows the primers used for the genotyping.

TABLE 8

| SEQ. | | |
|---|---|---|
| No. | names of primers | sequences |
| 24 | 5′ Hprt Tet- | atgggtgtatggg |
| | on genotype f | aagaggggagaga |
| 25 | 5′ Hprt Tet- | ttcctaccctcgtaa |
| | on genotype r | agaattcatggacgg |
| 26 | 3′ Hprt Tet- | GTGCCACTCCCACTG |
| | on genotype f | TCCTTTCCTA |
| 27 | 3′ Hprt Tet- | aatcatgttaaactgg |
| | on genotype r | aggagggtagtggtg |

### (Results and Discussions)

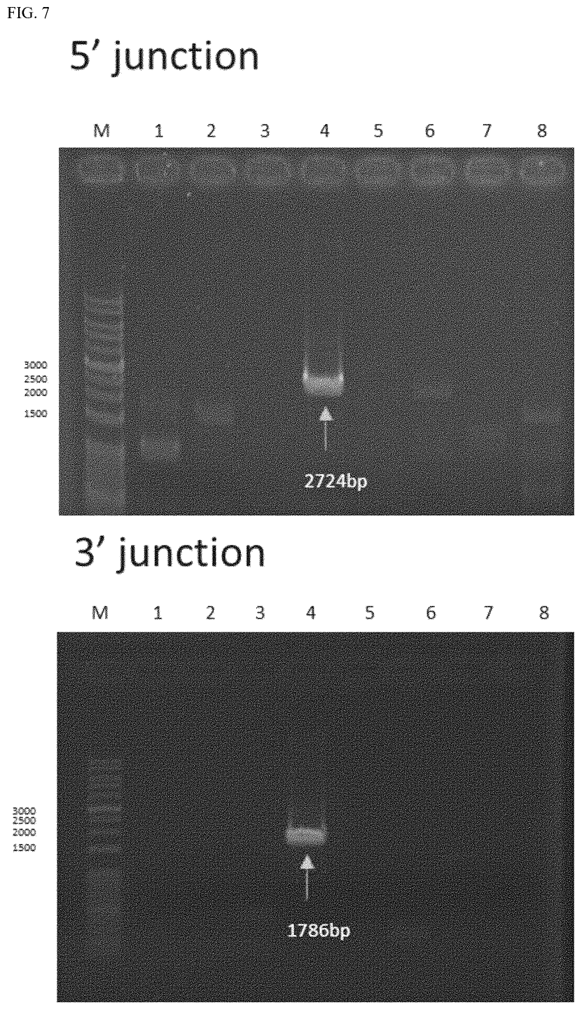

The results of the genotyping showed that an amplified product was observed at a correct location on both the 5′ side and the 3′ side in one individual. The results are shown in FIG. 7. As seen above, it was confirmed that Tet-on-Bax-IRES-mem-AcGFP had been inserted into an intended region.

### 3. Sex Selection Test Using Genetically Recombinant Male Mice Having Tet-On-Bax on X Chromosome

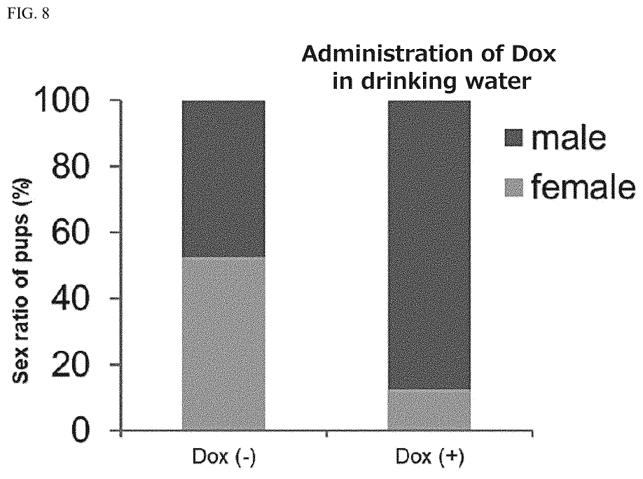

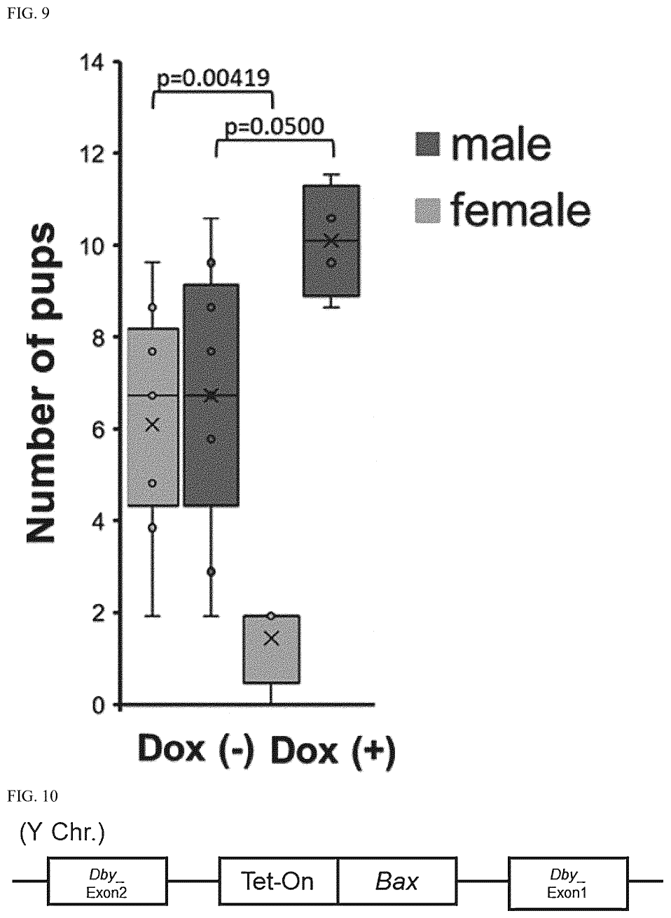

The obtained genetically recombinant male mice and wild-type female mice of the same species were mated at 20:00, and 12 hours later, at 8:00 a.m., the plug was checked to confirm that mating had occurred. To female mice which were confirmed to have mated, tap water mixed with 2 mg/mL of doxycycline (Dox) and 5% of sucrose was administered in drinking water. The control drank tap water. The mixing of sucrose is intended to relieve rejection of the drinking water due to bitterness of doxycycline. For the offsprings to each female mouse, the numbers of male and female offsprings were calculated at the weaning age. The results are shown in FIGS. 8 and 9.

As shown in FIGS. 8 and 9, males and females constituted about 90% and about 10%, respectively, of offsprings from the mice that had drunk water containing 2 mg/mL of doxycycline (Dox).

The obtained genetically recombinant male mice and wild-type female mice of the same species were mated at 20:00, and 12 hours later, at 8:00 a.m., the plug was checked to confirm that mating had occurred. Fertilized eggs were extracted from the uterine tube of female mice which were confirmed to have mated, and the fertilized eggs were cultured in KSOM medium containing 100 ng/ml of doxycycline (Dox). At 96 hours after the fertilization, genotyping was performed on surviving fertilized eggs to identify males and females. The table below shows the results.

TABLE 9

| Sex ratio of embryos (%) | female | male |
|---|---|---|
| Dox(+) | 16.7 | 83.3 |

As shown in the table above, males and females constituted 83.3% and 16.7%, respectively, of fertilized eggs which survived after being cultured in KSOM medium containing 100 ng/ml of doxycycline (Dox).

### 4. Preparation of Genetically Recombinant Mice Having Tet-On-Bax on Y Chromosome

The present experiment was aimed at preparing genetically recombinant male mice having Tet-on-Bax on a Y chromosome. With reference to a prior study in which tdTomato is knocked in to a Y chromosome (Hirata et al., 2022, Experimental Animals, 71(1), 82), the intragenic regions of exon 1 and exon 2 in the Ddx3y gene (hereinafter, described as Dby) were selected as knock-in regions (FIG. 10).

In the present experiment, a technique was adopted in which a plasmid having Cas9 RNP (Cas9 ribonucleoprotein) with Cas9 protein and gRNA bound to each other and a homology arm at both ends of a gene cassette is microinjected into the pronucleus of a mouse fertilized egg. In the present experiment, a GFP gene was knocked in to Dby to confirm the knock-in efficiency. Further, genetically recombinant mice were prepared in which Tet-on-Bax was knocked in to Dby. Specific descriptions are given below.

### 4-1. Knock-in of GFP Gene to Dby Region

In the present experiment, a GFP gene was knocked in to the Dby region (intragenic regions of exon 1 and exon 2 in Ddx3y gene) to confirm the knock-in efficiency and the possibility of occurrence of non-specific insertion. Specific experimental methods and results are shown below.

### Materials and Methods

1) Preparation of gRNA

Table 10 shows the sequence of gRNA prepared with reference to a prior study (Hirata et al., 2022, Experimental Animals, 71(1), 82.). The gRNA was prepared by mixing 18 μM of crRNA (IDT) and 18 μl of tracrRNA (IDT) using Nuclease-free Duplex Buffer, followed by treatment at 95° C. for 15 minutes and at 25° C. for 5 minutes.

TABLE 10

| SEQ. | | |
|---|---|---|
| No. | gRNA | sequences |
| 28 | Dby | tggcagctcactcccaactctgg |

### 2) Preparation of Cas9 RNP

Cas9 RNP was prepared by performing mixing in 5 μl of Opti-MEM (Life Technologies Corporation) containing 100 ng/μl of gRNA and 100 ng/μl of Cas9 Nuclease protein NLS (NIPPON GENE CO., LTD.).

3) Construction of pBS-DbyArm-CAG-EGFP Vector

The Dby region (intragenic regions of exon 1 and exon 2 of Ddx3y gene) on an Y chromosome was amplified from genomic DNA of the ovary of mice by performing PCR using KOD-plus-neo (TOYOBO CO., LTD.). The PCR product was inserted into the prepared pBS-SK(−)-FLAG vector using In-Fusion HD Cloning Kit, and then transformed into Escherichia coli DH5u to prepare a pBS-DbyArm vector. Next, the CAG-EGFP sequence was amplified from pR26 CAG/GFP Asc vector (Addgene) by performing PCR using KOD-plus-neo, followed by preparation of pBS-DbyArm-CAG-EGFP using Gibson Assembly Master Mix (NEW ENGLAND Biolabs). Table 11 shows the primer sequences.

TABLE 11

| SEQ. | names of | |
|---|---|---|
| No. | primers | sequences |
| 29 | Dby-arm f | tgtgtgggcgttgtcctctgg |
| | | agtctctaactccagttccat |
| 30 | Dby-arm r | ccactgcagctccctttggga |
| | | gtgagctgccatgtacattct |
| 31 | CAG-EGFP to | agggagctgcagtggagtagg |
| | vector f | c |
| 32 | CAG-EGFP to | gacaacgcccacacaccaggt |
| | vector r | t |

### 4) Extraction of Egg Mother Cells, In Vitro Fertilization and Culture of Embryos

The same method as in the section “1. Suppression of generation of fertilized eggs by expression of Bax” was carried out.

### 5) Microinjection

Microinjection was performed on the male pronuclei of fertilized eggs 6 to 8 hours after the fertilization.

### 6) Genotyping of Fertilized Eggs

Genotyping was performed on the fertilized eggs. Table 12 below shows the primers used.

TABLE 12

| SEQ. | names of | |
|---|---|---|
| No. | primers | sequences |
| 33 | Dby_genotype_1_f | ggcctaaagtttat |
| | | cacctgctagctg |
| 34 | Dby_genotype_1_r | cagatgctagaaa |
| | | gactggagttgcag |
| 35 | Dby_genotype_2_f | ggatccactagttc |
| | | tagagcggccgat |
| 36 | Dby_genotype_2_r | gaaccaaccagacg |
| | | accagaccctaga |

### (Results and Discussions)

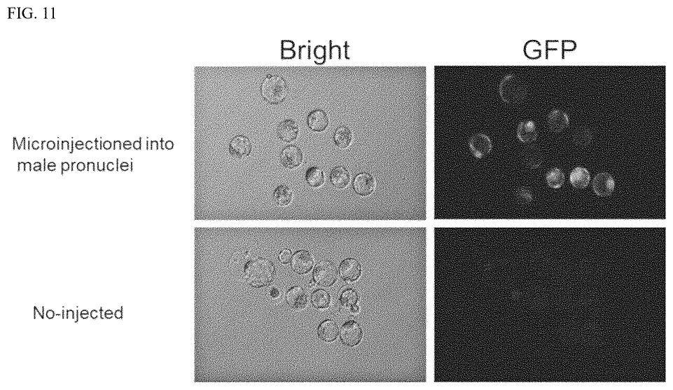

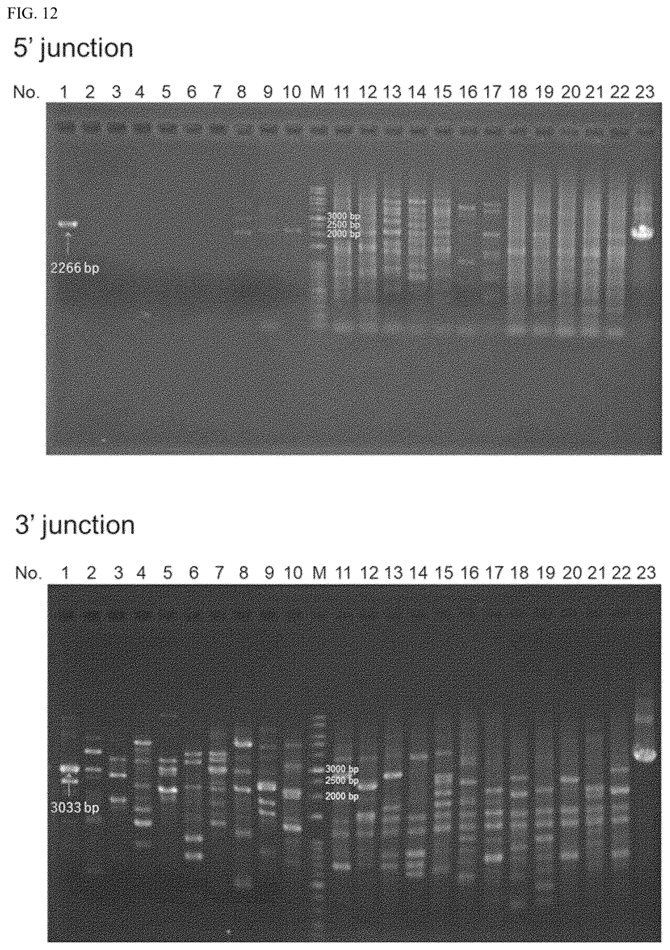

GFP fluorescence was observed for 10 out of the 22 fertilized eggs into which pBS-Dby-arm-CAG-EGFP and Cas9 RNP had been co-injected. The results are shown in FIG. 11. It was confirmed by genotyping that in one of the 10 fertilized eggs, knock-in to an appropriate region had been performed. The results are shown in FIG. 12.

The knock-in efficiency was 4.5% when simply calculated. This time, DNA was microinjected into the male pronucleus. Therefore, if a half of the relevant fertilized eggs are males, the male pronuclei of these fertilized eggs have no Y chromosome. Given the above, the knock-in efficiency may be further high. In nine out of the ten fertilized eggs where fluorescent was observed, a band with an appropriate size was not obtained. This indicates that non-specific insertion is considered to have occurred in the homologous recombination method adopted this time, and therefore, in future preparation of genetically recombinant mice in which Tet-on-Bax is knocked in, it is necessary to confirm that non-specific insertion does not occur.

### 4-2. Preparation of Genetically Recombinant Mice Having Tet-On-Bax on Y Chromosome

In the present experiment, genetically recombinant male mice having Tet-on-Bax in the intragenic regions of exon 1 and exon 2 in the Ddx3y gene of a Y chromosome were prepared. Specific experimental methods and results are shown below.

### Materials and Methods

1) Preparation of pBS-DbyArm-Tet-on-Bax

Using, as a template, the pBS-DbyArm-CAG-EGFP prepared in accordance with the section “4-1. Knock-in of GFP gene to Dby region”, a region excluding a CAG-EGFP cassette was amplified using KOD-plus neo (TOYOBO CO., LTD.), and the gene cassette region of the pTet-on-Bax prepared according to the section “1. Suppression of generation of fertilized eggs by expression of Bax” was amplified using KOD-plus neo. The two DNA fragments were bound with Gibson Assembly Master Mix (NEW ENGLAND BioLabs Inc.) to prepare pBS-DbyArm-Tet-on-Bax.

### 2) Collection of Egg Mother Cells, In Vitro Fertilization and Culture of Embryos

The same method as in the section “1. Suppression of generation of fertilized eggs by expression of Bax” was carried out.

### 3) Microinjection

The same microinjection method as in the section “4-1. Knock-in of GFP gene to Dby region” was carried out. The microinjection was performed on the male pronucleus.

### 4) Transplantation of Embryos

After the microinjection, the fertilized eggs were cultured for 96 hours, and then transplanted into the uterus of pseudopregnant ICR mice using mNSET Device for Mice 60010 (ParaTechs). In the transplantation, the fertilized eggs of wild-type mice were mixed with the fertilized eggs in which microinjection had been performed, and the mixed fertilized eggs were transplanted at the same time such that the total number of transplants was 20.

### 5) Genotyping of Offsprings

The fingers of 1.5-week-old offsprings were cut, treated with Proteinase K for 24 hours, and then extracted with phenol to obtain genomic DNA. With the obtained genomic DNA as a template, PCR was performed using KOD-FX neo (TOYOBO CO., LTD.), followed by genotyping. Table 13 shows the primers used for the genotyping.

TABLE 13

| SEQ. | names of | |
|---|---|---|
| No. | primers | sequences |
| 37 | Dby_genotype_3_f | ggcctaaagtttat |
| | | cacctgctagctg |
| 38 | Dby_genotype_3_r | ttcctaccctcgtaa |
| | | agaattcatggacgg |
| 39 | Dby_genotype_4_f | cttgacatgctccc |
| | | cgggtaaatctcc |
| 40 | Dby_genotype_4_r | gaaccaaccagacg |
| | | accagaccctaga |

### (Results and Discussions)

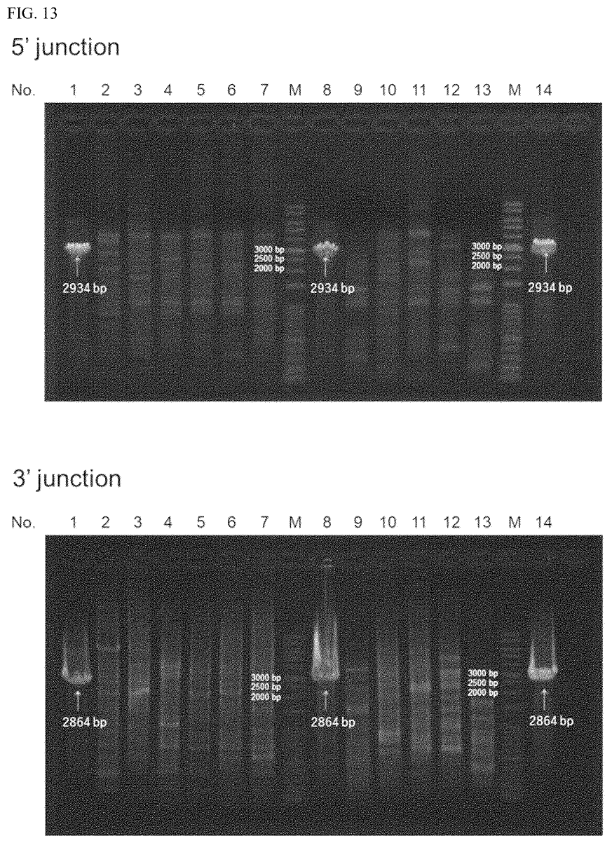

The results of the genotyping showed that an amplified product was observed at a correct location on both the 5′ side and the 3′ side in 3 out of 14 individuals. It can be said that genetically recombinant mice were obtained with an efficiency of 21.4%. The results are shown in FIG. 13.

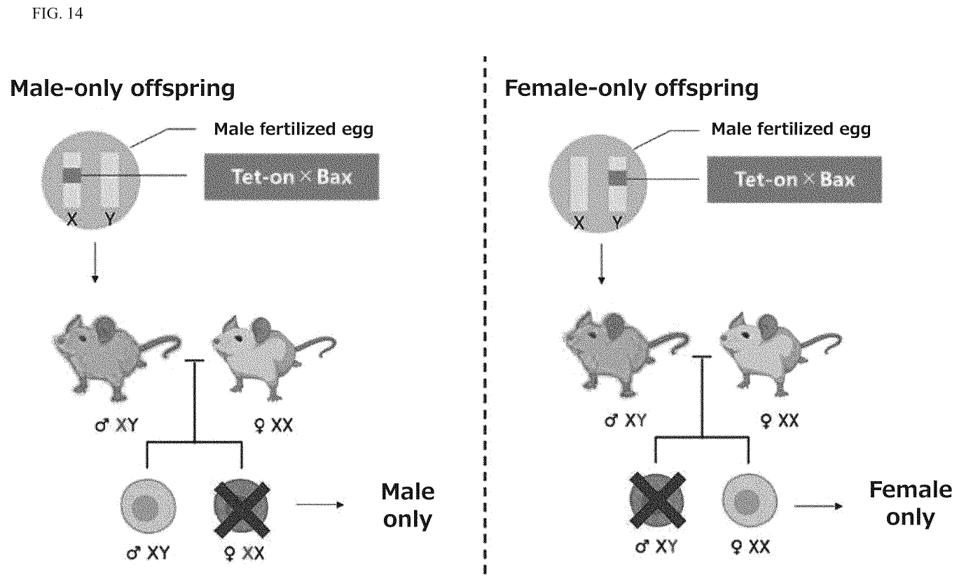

A schematic diagram of the action of sex selection mice is shown (FIG. 14).

### SEQUENCE LISTING

1. A male transgenic non-human animal in which one of an X chromosome and a Y chromosome comprises an exogenous embryonic death induction gene operatively connected to a drug-responsive promoter.

2. The male transgenic non-human animal according to claim 1, wherein the drug-responsive promoter is selected from the group consisting of a tetracycline-responsive promoter, a cumate-responsive promoter, a hormone-responsive promoter and an RSL1-responsive promoter.

3. A method for acquiring a pregnant non-human animal, comprising the step of mating a male transgenic non-human animal in which an X chromosome or a Y chromosome comprises an exogenous embryonic death induction gene operatively connected to a drug-responsive promoter and a female non-human animal of the same species which is free of an exogenous embryonic death induction gene.

4. A method for selecting a fertilized egg free of an exogenous embryonic death induction gene, comprising the step of treating a fertilized egg of the pregnant non-human animal acquired by the method according to claim 3 with a drug to which the promoter responds.

5. The method for selecting a fertilized egg according to claim 4, wherein the step of treatment with a drug is a step of administering the drug to the pregnant non-human animal, or a step of collecting a fertilized egg from the pregnant non-human animal and culturing the fertilized egg in a medium containing the drug.

6. A method for producing a male non-human animal, comprising the steps of:
(A1) mating a male transgenic non-human animal in which an X chromosome comprises an exogenous embryonic death induction gene operatively connected to a drug-responsive promoter and a female non-human animal of the same species which is free of an exogenous embryonic death induction gene; and
(B1) treating a fertilized egg of a pregnant non-human animal obtained by the step (A1) with the drug.

7. The method for producing a male non-human animal according to claim 6, wherein the step (B1) is a step of administering the drug to the pregnant non-human animal obtained by the step (A1), or a step of collecting a fertilized egg from the pregnant non-human animal obtained by the step (A1), and culturing the fertilized egg in a medium containing the drug.

8. A method for producing a female non-human animal, comprising the steps of:
(A2) mating a male transgenic non-human animal in which a Y chromosome comprises an exogenous embryonic death induction gene operatively connected to a drug-responsive promoter and a female non-human animal of the same species which is free of an exogenous embryonic death induction gene; and
(B2) treating a fertilized egg of a pregnant non-human animal obtained by the step (A2) with the drug.

9. The method for producing a female non-human animal according to claim 8, wherein the step (B2) is a step of administering the drug to the pregnant non-human animal obtained by the step (A2), or a step of collecting a fertilized egg from the pregnant non-human animal obtained by the step (A2), and culturing the fertilized egg in a medium containing the drug.

10. A method for producing a male non-human animal, comprising the steps of:
(A3) performing in vitro fertilization using sperm of a male transgenic non-human animal in which an X chromosome comprises an exogenous embryonic death induction gene operatively connected to a drug-responsive promoter and egg mother cells of a female non-human animal of the same species which is free of an exogenous embryonic death induction gene;
(B3) treating a fertilized egg obtained by the step (A3) with the drug; and
(C3) implanting a fertilized egg having survived the step (B3) in a foster mother.

11. A method for producing a female non-human animal, comprising the steps of:
(A4) performing in vitro fertilization using sperm of a male transgenic non-human animal in which a Y chromosome comprises an exogenous embryonic death induction gene operatively connected to a drug-responsive promoter and egg mother cells of a female non-human animal of the same species which is free of an exogenous embryonic death induction gene;
(B4) treating a fertilized egg obtained by the step (A4) with the drug; and
(C4) implanting a fertilized egg having survived the step (B4) in a foster mother.

12. An embryonic death induction gene expression system comprising:
a first expression cassette comprising a drug-responsive promoter sequence, and an embryonic death induction gene arranged downstream of the promoter sequence; and
a second expression cassette comprising a promoter sequence functioning in a fertilized egg, and a sequence encoding a drug-controlled trans-activator arranged downstream of the promoter sequence.

13. The embryonic death induction gene expression system according to claim 12, wherein the first expression cassette is held in a first vector, and the second expression cassette is held in a second vector.

14. The embryonic death induction gene expression system according to claim 12, wherein the first expression cassette and the second expression cassette are held in one vector.

15. A method for producing a male transgenic non-human animal for sex selection, comprising introducing the embryonic death induction gene expression system according to claim 12 into a fertilized egg, culturing an embryo, and then transplanting the embryo into a uterus of a pseudopregnant non-human animal to obtain an offspring.
